(19) United States
(12) Patent Application Publication
(10) Pub. No.: US 2025/0316389 A1
(43) Pub. Date: Oct. 9, 2025

(54) BIOMARKER-BASED RISK MODEL TO PREDICT DEATH AND PERSISTENT MULTIPLE ORGAN DYSFUNCTION SYNDROME IN PEDIATRIC SEPTIC SHOCK

(71) Applicant: Children's Hospital Medical Center, Cincinnati, OH (US)

(72) Inventors: Mihir ATREYA, Cincinnati, OH (US); Hector R. WONG, Cincinnati, OH (US)

(73) Assignee: Children's Hospital Medical Center, Cincinnati, OH (US)

(21) Appl. No.: 18/863,490

(22) PCT Filed: May 31, 2023
(86) PCT No.: PCT/US2023/067716

§ 371 Date: Nov. 6, 2024

Related U.S. Application Data
(60) Provisional application No. 63/347,944, filed on Jun. 1, 2022.

(51) Int. Cl. G16H 50/30; G16H 10/40; G16H 20/10

(52) U.S. Cl. CPC G16H 50/30; G16H 10/40; G16H 20/10

(57) ABSTRACT

Methods and compositions disclosed herein generally relate to methods of identifying, validating, and measuring clinically relevant, quantifiable biomarkers of diagnostic and therapeutic responses for blood, vascular, cardiac, and respiratory tract dysfunction, particularly as those responses relate to septic shock in pediatric patients. Certain aspects of the disclosure relate to identifying one or more biomarkers associated with septic shock in pediatric patients in combination with one or more endothelial-derived biomarkers, receiving a sample from a pediatric patient having at least one indication of septic shock, then quantifying from the sample an amount of said biomarkers, wherein the level of said biomarker correlates with a predicted outcome.

### CROSS REFERENCE TO RELATED APPLICATION

The present application is a 35 U.S.C. § 371 national stage application from PCT/US23/67716, filed May 31, 2023, which claims the benefit of priority under 35 U.S.C. § 119(e) to U.S. Provisional Application No. 63/347,944, PEDIATRIC SEPSIS MULTIPLE ORGAN DYSFUNCTION SYNDROME RISK PREDICTION MODEL, filed on Jun. 1, 2022, which is currently co-pending herewith and which is incorporated by reference in its entirety.

### STATEMENT REGARDING FEDERALLY-SPONSORED RESEARCH

This invention was made with government support under Grant No. R35 GM126943 awarded by the National Institutes of Health. The government has certain rights in the invention.

### FIELD

The disclosure herein generally relates to the identification and validation of clinically relevant, quantifiable biomarkers of diagnostic and therapeutic responses for blood, vascular, cardiac, and respiratory tract dysfunction, in particular septic shock, and in more particular aspects to integration of whole blood/leukocyte and endothelial-derived biomarkers to predict sepsis associated organ dysfunctions among children (pediatric patients).

### BACKGROUND

Septic shock is a leading cause of morbidity and mortality among children admitted to pediatric intensive care units (PICU) [1]. Patients with multiple organ dysfunction syndrome (MODS) disproportionately represent those with poor outcomes [1]. In addition, patients with persistent MODS are at highest risk of early [1] and late mortality [2], new medical device acquisition [3], and long-term neurocognitive impairment [4]. The current standard of care, namely antibiotics and intensive care [5], although appropriate for most patients, may be insufficient for those with MODS. Early identification of patients who may benefit from timely institution of targeted therapeutics remains a challenge.

Clinical and biological heterogeneity among septic patients has long confounded efforts to develop efficacious therapeutics [6]. Precision medicine approaches offer potential solutions to sift through this underlying heterogeneity [7].

Organ dysfunctions in sepsis is partly driven by interaction of activated leukocytes with the endothelium, with subsequent dysregulation of cascades of inflammation and coagulation, and resultant tissue hypoperfusion [9,10]. Despite this biological interplay, most studies of prognostic biomarkers in septic shock have considered the roles of these compartments separately rather than together. The serum Pediatric Sepsis Biomarker Risk Model (PERSEVERE), based on agnostic whole blood and leukocyte gene-expression studies [11,12], has been prospectively validated to estimate baseline risk of sepsis mortality [13-15]. More recently, it has been used to predict sepsis-associated acute kidney injury and myocardial dysfunction [16,17], and pediatric acute respiratory distress syndrome [18]. In parallel, markers of endothelial dysfunction have been variably correlated with mortality and organ dysfunctions in adult [19] and pediatric sepsis [20]. The prognostic capabilities of the latter to determine clinical outcomes are yet to be validated.

### SUMMARY

Embodiments of the disclosure relate to computer-implemented methods of classifying a patient with septic shock as high risk of multiple organ dysfunction syndrome (MODS) and/or mortality or other than high risk of MODS and/or mortality, the methods including: receiving a sample from a pediatric patient with septic shock at a first time point; analyzing the sample to determine expression levels of two or more biomarkers selected from IL-8, HSP70, ICAM-1, Thrombomodulin, Angpt-2/Angpt-1, and Angpt-2/Tie-2; determining whether the expression levels of each of the at least two biomarkers are greater than a respective cut-off biomarker concentration; and classifying the patient as high risk of multiple organ dysfunction syndrome (MODS) and/or mortality, or other than high risk of MODS and/or mortality, based on the determination of whether the expression levels of each of the at least two biomarkers are greater than the respective cut-off expression level. In some embodiments, a classification other than high risk includes a classification of low risk or intermediate risk.

In some embodiments, a classification of high risk of MODS and/or mortality includes: a) a non-elevated level of ICAM-1, and an elevated level of IL-8; b) an elevated level of ICAM-1, a non-elevated level of Angpt-2/Tie-2, and an elevated level of Thrombomodulin; or c) an elevated level of ICAM-1, and an elevated level of Angpt-2/Tie-2; and a classification of other than high risk of MODS and/or mortality includes: d) a non-elevated level of ICAM-1, a non-elevated level of IL-8, a non-elevated level of Angpt-2/Angpt-1, and a non-elevated level of HSP70; e) a non-elevated level of ICAM-1, a non-elevated level of IL-8, a non-elevated level of Angpt-2/Angpt-1, and an elevated level of HSP70; f) a non-elevated level of ICAM-1, a non-elevated level of IL-8, and an elevated level of Angpt-2/Angpt-1; or g) an elevated level of ICAM-1, a non-elevated level of Angpt-2/Tie-2, and a non-elevated level of Thrombomodulin.

In some embodiments, biomarker expression levels can be determined by quantification of serum protein biomarker concentrations. In some embodiments, biomarker expression levels can be determined by concentrations and/or by cycle threshold (CT) values.

In some embodiments, the determined biomarker expression levels include expression levels of one or more pairs of biomarkers selected from ICAM-1 and IL-8; ICAM-1 and Angpt-2/Tie-2; Angpt-2/Tie-2 and Thrombomodulin; IL-8 and Angpt-2/Angpt-1; and Angpt-2/Angpt-1 and HSP70. In some embodiments, the determined biomarker expression levels include expression levels of three or more selected from IL-8, HSP70, ICAM-1, Thrombomodulin, Angpt-2/Angpt-1, and/or Angpt-2/Tie-2. In some embodiments, the determined biomarker expression levels include expression levels of a trio of biomarkers selected from ICAM-1, IL-8, and Angpt-2/Angpt-1; IL-8, Angpt-2/Angpt-1, and HSP70; and ICAM-1, Angpt-2/Tie-2, and Thrombomodulin. In some embodiments, the determined biomarker expression levels include expression levels of IL-8, HSP70, ICAM-1, Thrombomodulin, Angpt-2/Angpt-1, and Angpt-2/Tie-2.

In some embodiments, biomarker levels are determined by serum protein biomarker concentration, and: a) an elevated level of IL-8 corresponds to a serum IL-8 concentration greater than 3.66 log 10 fold change; b) an elevated level of HSP70 corresponds to a serum HSP70 concentration greater than 6.32 log 10 fold change; c) an elevated level of ICAM-1 corresponds to a serum ICAM-1 concentration greater than 5.89 log 10 fold change; d) an elevated level of Thrombomodulin corresponds to a serum Thrombomodulin concentration greater than 3.94 log 10 fold change; e) an elevated level of Angpt-2/Angpt-1 ratio corresponds to a serum Angpt-2/Angpt-1 ratio greater than 0.45; and f) an elevated level of Angpt-2/Tie-2 corresponds to a serum Angpt-2/Tie-2 ratio greater than 1.06.

In some embodiments, the determination of whether the levels of the at least two biomarkers are non-elevated above a cut-off level includes applying the biomarker expression level data to a decision tree including the two or more biomarkers. In some embodiments, the biomarker expression level data is applied to the decision tree of FIG. 9.

In some embodiments, MODS includes cardiovascular, respiratory, renal, hepatic, hematologic, and/or neurologic dysfunction. In some embodiments, MODS includes cardiovascular dysfunction. In some embodiments, MODS includes dysfunction in one or more organs selected from heart, lungs, kidneys, liver, blood, and brain. In some embodiments, high risk of MODS and/or mortality by day 7 of septic shock or other than high risk of MODS and/or mortality by day 7 of septic shock can be determined.

In some embodiments, the classification can be combined with one or more patient demographic data and/or clinical characteristics and/or results from other tests or indicia of septic shock and/or one or more additional biomarkers. In some embodiments, the one or more additional biomarkers can include C-C Chemokine ligand 3 (CCL3), C-C Chemokine ligand 4 (CCL4), Granzyme B (GZMB), Interleukin-1 α (IL-1a), Matrix metallopeptidase 8 (MMP8), Angiopoietin-1 (Angpt-1), Angiopoietin-2 (Angpt-2), Tyrosine kinase with immunoglobulin-like loops and epidermal growth factor homology domains-2 (Tie-2), Vascular cell adhesion molecule-1 (VCAM-1), P-selectin, E-selectin, and Platelet and endothelial cell adhesion molecule-1 (PECAM-1). In some embodiments, the patient demographic data and/or clinical characteristics and/or results from other tests or indicia of septic shock include at least one selected from the septic shock causative organism, the presence or absence or chronic disease, and/or the age, gender, race, and/or co-morbidities of the patient. In some embodiments, wherein the classification can be combined with one or more additional population-based risk scores. In some embodiments, the one or more population-based risk scores can include at least one of Pediatric Sepsis Biomarker Risk Model (PERSEVERE), Pediatric Risk of Mortality (PRISM), PRISM III, Pediatric Index of Mortality (PIM), and/or Pediatric Logistic Organ Dysfunction (PELOD).

In some embodiments, the sample can be obtained within the first hour of presentation with septic shock. In some embodiments, the sample can be obtained within the first 24 hours of presentation with septic shock. In some embodiments, the sample can be obtained within the first 48 hours of presentation with septic shock. In some embodiments, the sample can be obtained within the first 72 hours of presentation with septic shock. In some embodiments, the sample can be obtained within the first 24-48 hours of presentation with septic shock. In some embodiments, the sample can be obtained within the first 48-72 hours of presentation with septic shock.

In some embodiments of the methods, a treatment including one or more high risk therapy to a patient that is classified as high risk, or administering a treatment excluding a high risk therapy to a patient that is not high risk, or to provide a method of treating a pediatric patient with septic shock, can be administered. In some embodiments, the one or more high risk therapy includes at least one of biological and/or immune enhancing therapy, extracorporeal membrane oxygenation/life support, plasmapheresis, pulmonary artery catheterization, high volume continuous hemofiltration, adjuvant hemoperfusion, adjuvant hemoperfusion, extracorporeal hemadsorption, and/or plasma filtration and/or adsorption therapies. In some embodiments, the biological and/or immune enhancing therapy includes administration of GM-CSF, Interleukin-1 receptor antagonist, Interleukin-1 receptor antagonist, Interleukin-6 antagonist, anti-PD-1, recombinant thrombomodulin, Angiopoietin-2 inhibitors, and/or Angiopoietin-1 or Tie-2 agonist, and/or anti-PD-1.

In some embodiments, the patient can be enrolled in a clinical trial. In some embodiments, the patient enrolled in a clinical trial is classified as high risk. Some embodiments of the methods include prognostic enrichment through enrollment of the high risk patient in the clinical trial. In some embodiments, a treatment including one or more high risk therapy to the patient in the clinical trial can be administered.

Some embodiments of the methods include improving an outcome in a pediatric patient with septic shock. In some embodiments, the methods include: receiving a second sample from the treated patient at a second time point; analyzing the second sample to determine expression levels of two or more biomarkers including IL-8, HSP70, ICAM-1, Thrombomodulin, Angpt-2/Angpt-1, and/or Angpt-2/Tie-2; determining whether the biomarker expression levels of each of the biomarkers are greater than a respective cut-off biomarker expression level; classifying the patient as high risk of multiple organ dysfunction syndrome (MODS) and/or mortality, or other than high risk of MODS and/or mortality, based on the determination of whether the expression levels of each of the biomarkers are greater than the respective cut-off expression level; maintaining the treatment being administered if the patient's high risk classification has not changed, or changing the treatment being administered if the patient's high risk classification has changed. In some embodiments, the second time point can be at least 18 hours after the first time point. In some embodiments, the second time point can be in the range of 24 to 96 hours, or longer, after the first time point. In some embodiments, the second time point can be about 1 day, 2 days, 3 days, or longer, after the first time point. In some embodiments, the second time point can be about 2 days after the first time point. In some embodiments, the first time point can be at day 1, wherein day 1 can be within 24 hours of a septic shock diagnosis, and the second time point can be at day 3. In some embodiments, the first time point can be within 24, 48, or 72 hours of a septic shock diagnosis, and the second time point can be 1, 2, or 3 days after the first time point.

In some embodiments, a patient classified as high risk after the second time point can be administered one or more high risk therapy. In some embodiments, the one or more high risk therapy includes at least one selected from the group consisting of biological and/or immune enhancing therapy, extracorporeal membrane oxygenation/life support, plasmapheresis, pulmonary artery catheterization, high volume continuous hemofiltration, adjuvant hemoperfusion, adjuvant hemoperfusion, extracorporeal hemadsorption, and/or plasma filtration and/or adsorption therapies. In some embodiments, the one or more high risk therapy includes a biological and/or immune enhancing therapy. In some embodiments, a patient not classified as high risk after the second time point can be administered a treatment excluding a high risk therapy. In some embodiments, the patient classified as high risk and administered one or more high risk therapy after the first time point can be not classified as high risk after the second time point.

In some embodiments, the methods are used as part of a companion diagnostic.

Further embodiments of the disclosure relate to diagnostic kits, tests, or arrays including a reporter hybridization probe, and a capture hybridization probe specific for each of two or more mRNA, DNA, or protein biomarkers selected from IL-8, HSP70, ICAM-1, Thrombomodulin, Angpt-2/Angpt-1, and Angpt-2/Tie-2. In some embodiments, the biomarkers can include three or more selected from IL-8, HSP70, ICAM-1, Thrombomodulin, Angpt-2/Angpt-1, and Angpt-2/Tie-2. In some embodiments, the biomarkers can include IL-8, HSP70, ICAM-1, Thrombomodulin, Angpt-2/Angpt-1, and Angpt-2/Tie-2. In some embodiments, the diagnostic kits, tests, or arrays further include a collection cartridge for immobilization of the hybridization probes. In some embodiments, the reporter and the capture hybridization probes include signal and barcode elements, respectively.

Further embodiments of the disclosure relate to apparatuses or processing devices suitable for detecting two or more biomarkers selected from IL-8, HSP70, ICAM-1, Thrombomodulin, Angpt-2/Angpt-1, and Angpt-2/Tie-2. In some embodiments, the biomarkers can include three or more selected from IL-8, HSP70, ICAM-1, Thrombomodulin, Angpt-2/Angpt-1, and Angpt-2/Tie-2. In some embodiments, the biomarkers can include IL-8, HSP70, ICAM-1, Thrombomodulin, Angpt-2/Angpt-1, and Angpt-2/Tie-2.

Further embodiments of the disclosure relate to compositions including a reporter hybridization probe, and a capture hybridization probe specific for each of two or more biomarkers selected from IL-8, HSP70, ICAM-1, Thrombomodulin, Angpt-2/Angpt-1, and Angpt-2/Tie-2. In some embodiments, the biomarkers can include three or more selected from IL-8, HSP70, ICAM-1, Thrombomodulin, Angpt-2/Angpt-1, and Angpt-2/Tie-2. In some embodiments, the biomarkers can include IL-8, HSP70, ICAM-1, Thrombomodulin, Angpt-2/Angpt-1, and Angpt-2/Tie-2.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. FIG. 1A. Chord diagram representing inter-relationship between individual organ dysfunctions on day 7 of septic shock. FIG. 1B. Correlogram between pairs of individual organ dysfunctions on day 7 of septic shock

FIG. 2. Area under the receiver operating characteristic (AUROC) curve for the 22 variable TreeNet® PERSEVEREnce model to estimate risk of death or day 7 MODS in children with septic shock.

FIG. 3. Area under the receiver operating characteristic (AUROC) curve for the 22 variable TreeNet® PERSEVEREnce models to estimate risk of persistent a) cardiovascular, b) respiratory, c) renal, d), hepatic, e) hematologic, and f) neurologic dysfunction on day 7 of pediatric septic shock.

FIG. 4. Relative variable importance, with respect to the top predictor variable, in the 22 variable TreeNet® PERSEVEREnce model to estimate risk of death or day 7 MODS in children with septic shock.

FIGS. 5A, 5B, and 5C. One Predictor Partial Dependence Plots in the 22 variable TreeNet® PERSEVEREnce model to estimate risk of death or day 7 MODS in children with septic shock. For each continuous predictor variable, the range of values is shown on the x-axis. The fitted half log odds of death or day 7 MODS is shown on y-axis.

FIGS. 6A-6F. Relative variable importance, with respect to the top predictor variable, in the 22 variable TreeNet® PERSEVEREnce model to estimate risk of various individual organ dysfunctions on day 7 in children with septic shock. FIG. 6A. Variable importance for predicting day 7 cardiovascular dysfunction. FIG. 6B. Variable importance for predicting day 7 respiratory dysfunction. FIG. 6C. Variable importance for predicting day 7 renal dysfunction. FIG. 6D. Variable importance for predicting day 7 hepatic dysfunction. FIG. 6E. Variable importance for predicting day 7 hematologic dysfunction. FIG. 6F. Variable importance for predicting day 7 neurologic dysfunction.

FIG. 7. Surface and contour plots showing the relationship between the fitted half log odds of death or day 7 MODS and the two-way interaction between IL-8 with ICAM-1 and between Thrombomodulin with Angpt-2/Angpt-1 ratio in children with septic shock.

FIG. 8. Area under the receiver operating characteristic (AUROC) curve for the 6 variable TreeNet® PERSEVEREnce model to estimate risk of death or day 7 MODS in children with septic shock.

FIG. 9. Classification and regression tree (CART®) tree to estimate the risk of death or day 7 MODS in children with septic shock. The classification tree includes Interleukin-8 (IL-8), Intercellular adhesion molecule-1 (ICAM-1), Angiopoietin-2/Angiopoietin-1, Angiopoietin-2/Tie-2, Heat Shock Protein 70 (HSP70) and Thrombomodulin (TM). The biomarkers concentrations are log(10) transformed and ratios of Angpt-2/Angpt-1 and Angpt-2/Tie-2 are shown. The root node shows all patients included in the derivation cohort, with and without day 7 MODS, and their respective rates. The decision rule criteria for each daughter node and the rates of patients with and without day 7 MODS is shown. TN-1 is the only low-risk zone (˜10.6% risk). TN-2, 3, and 5 are intermediate risk (32.7-52.6% risk). TN-4, 6, and 7 are high-risk with 60.7-89.3% risk of death or day 7 MODS).

FIG. 10 is a block diagram that illustrates a computer system 400, upon which embodiments of the present teachings may be implemented.

### DETAILED DESCRIPTION

All references cited herein are incorporated by reference in their entirety. Also incorporated herein by reference in their entirety include: U.S. Patent Application No. 61/595,996, BIOMARKERS OF SEPTIC SHOCK, filed on Feb. 7, 2012; U.S. Provisional Application No. 61/721,705, A MULTI-BIOMARKER-BASED OUTCOME RISK STRATIFICATION MODEL FOR ADULT SEPTIC SHOCK, filed on Nov. 2, 2012; International Patent Application No. PCT/US13/25223, A MULTI-BIOMARKER-BASED OUTCOME RISK STRATIFICATION MODEL FOR PEDIATRIC SEPTIC SHOCK, filed on Feb. 7, 2013; International Patent Application No. PCT/US13/25221, A MULTI-BIOMARKER-BASED OUTCOME RISK STRATIFICATION MODEL FOR ADULT SEPTIC SHOCK, filed on Feb. 7, 2013; U.S. Provisional Application No. 61/908,613, TEMPORAL PEDIATRIC SEPSIS BIOMARKER RISK MODEL, filed on Nov. 25, 2013; International Patent Application No. PCT/US14/067438, TEMPORAL PEDIATRIC SEPSIS BIOMARKER RISK MODEL, filed on Nov. 25, 2014; U.S. patent application Ser. No. 15/998,427, SEPTIC SHOCK ENDOTYPING STRATEGY AND MORTALITY RISK FOR CLINICAL APPLICATION, filed on Aug. 15, 2018; U.S. Provisional Application No. 62/616,646, TEMPORAL ENDOTYPE TRANSITIONS REFLECT CHANGING RISK AND TREATMENT RESPONSE IN PEDIATRIC SEPTIC SHOCK, filed on Jan. 12, 2018; International Application No. PCT/US2017/032538, SIMPLIFICATION OF A SEPTIC SHOCK ENDOTYPING STRATEGY FOR CLINICAL APPLICATIONS, filed on May 12, 2017; U.S. Provisional Application No. 62/335,803, SIMPLIFICATION OF A SEPTIC SHOCK ENDOTYPING STRATEGY FOR CLINICAL APPLICATIONS, filed on May 13, 2016; U.S. Provisional Application No. 62/427,778, SIMPLIFICATION OF A SEPTIC SHOCK ENDOTYPING STRATEGY FOR CLINICAL APPLICATIONS, filed on Nov. 29, 2016; U.S. Provisional Application No. 62/428,451, SIMPLIFICATION OF A SEPTIC SHOCK ENDOTYPING STRATEGY FOR CLINICAL APPLICATIONS, filed on Nov. 30, 2016; U.S. Provisional Application No. 62/446,216, SIMPLIFICATION OF A SEPTIC SHOCK ENDOTYPING STRATEGY FOR CLINICAL APPLICATIONS, filed on Jan. 13, 2017; U.S. patent application Ser. No. 16/539,128, SEPTIC SHOCK ENDOTYPING STRATEGY AND MORTALITY RISK FOR CLINICAL APPLICATION, filed on Aug. 13, 2019; U.S. Provisional Application No. 62/764,831, ENDOTYPE TRANSITIONS DURING THE ACUTE PHASE OF PEDIATRIC SEPTIC SHOCK REFLECT CHANGING RISK AND TREATMENT RESPONSE, filed on Aug. 15, 2018; U.S. Provisional Application No. 63/149,744, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2021; and International Patent Application No. PCT/US2022/016642, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2022.

Unless otherwise noted, terms are to be understood according to conventional usage by those of ordinary skill in the relevant art.

As used herein, the term “sample” encompasses a sample obtained and/or received from a subject or patient. The sample can be of any biological tissue or fluid. Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, tear fluid, or cells therefrom. Samples can also include sections of tissues such as frozen or fixed sections taken for histological purposes or micro-dissected cells or extracellular parts thereof. A sample to be analyzed can be tissue material from a tissue biopsy obtained and/or received by aspiration or punch, excision or by any other surgical method leading to biopsy or resected cellular material. Such a sample can comprise cells obtained and/or received from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” includes any form of measurement, and includes determining if an element is present or not. The terms “determining,” “measuring,” “evaluating,” “assessing” and “assaying” can be used interchangeably and can include quantitative and/or qualitative determinations.

As used herein, the term “monitoring” with reference to septic shock refers to a method or process of determining the severity or degree of septic shock or stratifying septic shock based on risk and/or probability of mortality. In some embodiments, monitoring relates to a method or process of determining the therapeutic efficacy of a treatment being administered to a patient.

As used herein, “outcome” can refer to an outcome studied. In some embodiments, “outcome” can refer to organ dysfunction and/or death after septic shock. In some embodiments, “outcome” can refer to two or more organ dysfunctions or death by day 7 of septic shock. In some embodiments, “outcome” can refer to day 7 cardiovascular, respiratory, renal, hepatic, hematologic, and neurologic dysfunction.

In some embodiments, “outcome” can refer to 28-day survival/mortality. The importance of survival/mortality in the context of pediatric septic shock is readily evident. The common choice of 28 days was based on the fact that 28-day mortality is a standard primary endpoint for interventional clinical trials involving critically ill patients. In some embodiments, an increased risk for a poor outcome indicates that a therapy has had a poor efficacy, and a reduced risk for a poor outcome indicates that a therapy has had a good efficacy. In some embodiments, “outcome” can refer to resolution of organ failure after 14 days or 28 days or limb loss. Although mortality/survival is obviously an important outcome, survivors have clinically relevant short- and long-term morbidities that impact quality of life, which are not captured by the dichotomy of “alive” or “dead.” In the absence of a formal, validated quality of life measurement tool for survivors of pediatric septic shock, resolution of organ failure can be used as a secondary outcome measure. For example, the presence or absence of new organ failure over one or more timeframes can be tracked. Patients having organ failure beyond 28 days are likely to survive with significant morbidities having negative consequences for quality of life. Organ failure is generally defined based on published and well-accepted criteria for the pediatric population [21]. Specifically, cardiovascular, respiratory, renal, hepatic, hematologic, and neurologic failure can be tracked. In addition, limb loss can be tracked as a secondary outcome. Although limb loss is not a true “organ failure,” it is an important consequence of pediatric septic shock with obvious impact on quality of life.

As used herein, “outcome” can also refer to complicated course. Complicated course as defined herein relates to persistence of two or more organ failures at day seven of septic shock or 28-day mortality.

As used herein, the terms “predicting outcome” and “outcome risk stratification” with reference to septic shock refers to a method or process of prognosticating a patient's risk of a certain outcome. In some embodiments, predicting an outcome relates to monitoring the therapeutic efficacy of a treatment being administered to a patient. In some embodiments, predicting an outcome relates to determining a relative risk of an adverse outcome (e.g. complicated course) and/or mortality. In some embodiments, the predicted outcome is associated with administration of a particular treatment or treatment regimen. Such adverse outcome risk and/or mortality can be high risk, moderate risk, moderate-high risk, moderate-low risk, or low risk. Alternatively, such adverse outcome risk can be described simply as high risk or low risk, corresponding to high risk of adverse outcome (e.g. complicated course) and/or mortality probability, or high likelihood of therapeutic effectiveness, respectively. In some embodiments of the present disclosure, adverse outcome risk can be determined via the biomarker-based MODS and/or mortality risk stratification as described herein. In some embodiments, predicting an outcome relates to determining a relative risk of MODS and/or mortality. Such mortality risk can be high risk, moderate risk, moderate-high risk, moderate-low risk, or low risk. Alternatively, such mortality risk can be described simply as high risk or low risk, corresponding to high risk of death or high likelihood of survival, respectively. As related to the terminal nodes of the decision trees described herein, a “high risk terminal node” corresponds to an increased probability of adverse outcome (e.g. complicated course) and/or mortality according to a particular treatment or treatment regimen, whereas a “low risk terminal node” corresponds to a decreased probability of adverse outcome (e.g. complicated course) and/or mortality according to a particular treatment or treatment regimen.

As used herein, the term “high risk clinical trial” refers to one in which the test agent has “more than minimal risk” (as defined by the terminology used by institutional review boards, or IRBs). In some embodiments, a high risk clinical trial is a drug trial.

As used herein, the term “low risk clinical trial” refers to one in which the test agent has “minimal risk” (as defined by the terminology used by IRBs). In some embodiments, a low risk clinical trial is one that is not a drug trial. In some embodiments, a low risk clinical trial is one that that involves the use of a monitor or clinical practice process. In some embodiments, a low risk clinical trial is an observational clinical trial.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” refers to any member of the animal kingdom. In some embodiments, a subject is a human patient. In some embodiments, a subject is a pediatric patient. In some embodiments, a pediatric patient is a patient under 18 years of age, while an adult patient is 18 or older. Unless stated otherwise, the terms “patient” or “child” (or “patients” or “children”) refer to a pediatric patient (i.e., under 18 years old).

As used herein, the terms “treatment,” “treating,” “treat,” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition.

As used herein, the term “marker” or “biomarker” refers to a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, the term “expression levels” refers, for example, to a determined level of biomarker expression. The term “pattern of expression levels” refers to a determined level of biomarker expression compared either to a reference (e.g. a housekeeping gene or inversely regulated genes, or other reference biomarker) or to a computed average expression value (e.g. in DNA-chip analyses). A pattern is not limited to the comparison of two biomarkers but is more related to multiple comparisons of biomarkers to reference biomarkers or samples. A certain “pattern of expression levels” can also result and be determined by comparison and measurement of several biomarkers as disclosed herein and display the relative abundance of these transcripts to each other.

As used herein, a “reference pattern of expression levels” refers to any pattern of expression levels that can be used for the comparison to another pattern of expression levels. In some embodiments of the disclosure, a reference pattern of expression levels is, for example, an average pattern of expression levels observed in a group of healthy or diseased individuals, serving as a reference group.

As used herein, the term “decision tree” refers to a standard machine learning technique for multivariate data analysis and classification. Decision trees can be used to derive easily interpretable and intuitive rules for decision support systems.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

Multiple organ dysfunction syndrome (MODS) is the final common pathway among children with septic shock and disproportionately contributes to and is a critical driver of sepsis morbidity and mortality in children. Current standard of sepsis care is likely insufficient for those with MODS.

However, tools are currently lacking to reliably identify patients who will have persistent MODS and can benefit from targeted therapies. Prognostic enrichment through biomarker-based risk stratification, can allow for identification of patients at high-risk of death or persistent organ dysfunction who can be targeted for enrollment in future clinical trials of sepsis therapeutics. Conversely, those deemed low-risk can receive standard care and not be subject to potentially harmful therapies [11].

Early identification of those at risk of death and persistent organ dysfunctions is necessary to enrich patients for future trials of sepsis therapeutics and to treat such patients in the future. Accordingly, the present disclosure describes research that was conducted to determine whether integration of endothelial dysfunction and PERSEVERE biomarkers measured on day 1 can reliably estimate risk of death or organ dysfunctions on day 7 in a large pediatric septic shock cohort.

The disclosure herein relates to the use of integrated serum endothelial dysfunction and PERSEVERE (Pediatric Sepsis Biomarker Risk Model) biomarkers measured on day 1 of sepsis to reliably estimate the risk of death or persistent organ dysfunctions on day 7 of septic shock in a pediatric patient. This newly derived risk model, termed herein as PERSEVEREnce, can allow for prognostic enrichment of those patients with a significant burden of organ dysfunctions in future pediatric trials of sepsis therapeutics.

### Determining MODS and Mortality Risk

Reliable risk stratification has numerous clinical applications. These include better-informed allocation of critical care resources, appropriate selection of patients for higher risk and more costly therapies, and for benchmarking outcomes. Additionally, risk stratification can serve as a prognostic enrichment tool to greatly enhance efficiency of clinical trials. Reliable risk stratification of patients with septic shock can be a challenging task due to significant patient heterogeneity.

The Pediatric Sepsis Biomarker Risk Model (PERSEVERE) for estimating baseline mortality risk in children with septic shock was previously derived and validated. PERSEVERE is based on a panel of 12 serum protein biomarkers measured from blood samples obtained during the first 24 hours of a septic shock diagnosis, selected from among 80 genes having an association with mortality risk in pediatric septic shock.

The PERSEVERE biomarkers were initially identified through discovery-oriented transcriptomic studies searching for genes having an association with mortality in pediatric septic shock. From among the 80 genes identified in these studies, the biomarkers to be considered for inclusion in PERSEVERE were selected using two simultaneous criteria. First, the gene should have a biologically plausible link to septic shock pathophysiology. Second, the protein transcribed from the gene can be readily measured in the blood compartment. While pragmatic, the selection criteria were limited by existing knowledge and paradigms of septic shock pathophysiology, and by technical considerations, leaving just 12 potential biomarkers for consideration. Consequently, 68 genes were left unconsidered, some of which might have the ability to improve upon the ability of PERSEVERE to estimate baseline mortality risk, and some of which might provide information about biological mechanisms and pathophysiology associated with mortality in septic shock.

Endothelial dysfunction markers were measured from day 1 serum among those with existing PERSEVERE data. A TreeNet® classification model was derived incorporating 22 clinical and biological variables to estimate risk. Based on relative variable importance, a simplified 6 biomarker model was developed thereafter.

Among 502 patients, 49 patients died before day 7 and 124 patients had persistence of MODS on day 7 of septic shock. Area under the receiver operator characteristic curve (AUROC) for the newly derived PERSEVEREnce model to predict death or day 7 MODS was 0.93 (0.91-0.95), with a summary AUROC of 0.80 (0.76-0.84) upon 10-fold cross validation. The simplified model, based on IL-8, HSP70, ICAM-1, Angpt2/Tie2, Angpt2/Angpt1, and Thrombomodulin performed similarly. Interaction between variables, namely ICAM-1 with IL-8 and Thrombomodulin with Angpt2/Angpt1, contributed to the models' predictive capabilities. Model performance varied when estimating risk of individual organ dysfunctions with AUROCS ranging between 0.91 to 0.97 and 0.68 to 0.89 in training and test sets respectively. As such, the newly derived PERSEVEREnce biomarker model reliably estimates the composite risk of death or persistent organ dysfunction on day 7 of septic shock in pediatric septic shock. This tool can be used for prognostic enrichment in future pediatric trials of sepsis therapeutics.

Thus, in a large cohort of critically ill children, PERSEVERE was integrated with endothelial markers to reliably estimate risk of death or persistent organ dysfunctions on day 7 of septic shock. PERSEVEREnce biomarkers can therefore facilitate prognostic enrichment of pediatric patients with organ dysfunctions in future pediatric trials of sepsis therapeutics and in sepsis treatment.

### Additional Patient Information

The demographic data, clinical characteristics, and/or results from other tests or indicia of septic shock specific to a pediatric patient with septic shock can affect the patient's outcome risk. Accordingly, such demographic data, clinical characteristics, and/or results from other tests or indicia of septic shock can be incorporated into the methods described herein which allow for stratification of individual pediatric patients in order to determine the patient's outcome risk. Such demographic data, clinical characteristics, and/or results from other tests or indicia of septic shock can also be used in combination with the methods described herein which allow for stratification of individual pediatric patients in order to determine the patient's outcome risk.

Such pediatric patient demographic data can include, for example, the patient's age, race, gender, and the like. In some embodiments, the biomarker-based MODS and/or mortality risk stratification described herein can incorporate or be used in combination with the patient's age, race, and/or gender to determine an outcome risk.

Such patient clinical characteristics and/or results from other tests or indicia of septic shock can include, for example, the patient's co-morbidities and/or septic shock causative organism, and the like.

Patient co-morbidities can include, for example, acute lymphocytic leukemia, acute myeloid leukemia, aplastic anemia, atrial and ventricular septal defects, bone marrow transplantation, caustic ingestion, chronic granulomatous disease, chronic hepatic failure, chronic lung disease, chronic lymphopenia, chronic obstructive pulmonary disease (COPD), congestive heart failure (NYHA Class IV CHF), Cri du Chat syndrome, cyclic neutropenia, developmental delay, diabetes, DiGeorge syndrome, Down syndrome, drowning, end stage renal disease, glycogen storage disease type 1, hematologic or metastatic solid organ malignancy, hemophagocytic lymphohistiocytosis, hepatoblastoma, heterotaxy, hydrocephalus, hypoplastic left heart syndrome, IPEX Syndrome, kidney transplant, Langerhans cell histiocytosis, liver and bowel transplant, liver failure, liver transplant, medulloblastoma, metaleukodystrophy, mitochondrial disorder, multiple congenital anomalies, multi-visceral transplant, nephrotic syndrome, neuroblastoma, neuromuscular disorder, obstructed pulmonary veins, Pallister Killian syndrome, Prader-Willi syndrome, requirement for chronic dialysis, requirement for chronic steroids, retinoblastoma, rhabdomyosarcoma, rhabdosarcoma, sarcoma, seizure disorder, severe combined immune deficiency, short gut syndrome, sickle cell disease, sleep apnea, small bowel transplant, subglottic stenosis, tracheal stenosis, traumatic brain injury, trisomy 18, type 1 diabetes mellitus, unspecified brain tumor, unspecified congenital heart disease, unspecified leukemia, VATER Syndrome, Wilms tumor, and the like. Any one or more of the above patient co-morbidities can be indicative of the presence or absence of chronic disease in the patient. Septic shock causative organisms can include, for example, Acinetobacter baumannii, Adenovirus, Bacteroides species, Candida species, Capnotyophaga jenuni, Cytomegalovirus, Enterobacter cloacae, Enterococcus faecalis, Escherichia coli, Herpes simplex virus, Human metapneumovirus, Influenza A, Klebsiella pneumonia, Micrococcus species, mixed bacterial infection, Moraxella catarrhalis, Neisseria meningitides, Parainfluenza, Pseudomonas species, Serratia marcescens, Staphylococcus aureus, Streptococcus agalactiae, Streptococcus milleri, Streptococcus pneumonia, Streptococcus pyogenes, unspecified gram negative rods, unspecified gram positive cocci, and the like.

In some embodiments, the biomarker-based MODS and/or mortality risk stratification as described herein can incorporate the patient's co-morbidities to determine an outcome risk and/or mortality probability. In some embodiments, the biomarker-based MODS and/or mortality risk stratification as described herein can incorporate the patient's septic shock causative organism to determine an outcome risk and/or mortality probability.

In some embodiments, the biomarker-based MODS and/or mortality risk stratification as described herein can be used in combination with the patient's co-morbidities to determine an outcome risk and/or mortality probability. In some embodiments, the biomarker-based MODS and/or mortality risk stratification as described herein can be used in combination with the patient's septic shock causative organism to determine an outcome risk and/or mortality probability.

### PERSEVERE, PERSEVERE II, and Other Population-Based Risk Scores

As mentioned previously, the PERSEVERE model for estimating baseline mortality risk in children with septic shock was previously derived and validated. PERSEVERE is based on a panel of 12 serum protein biomarkers measured from blood samples obtained during the first 24 hours of a septic shock diagnosis, selected from among 80 genes having an association with mortality risk in pediatric septic shock. Of those 12 serum biomarkers, the derived and validated PERSEVERE model is based on Interleukin-8 (IL-8), Heat shock protein 70 kDA (HSP70), C-C Chemokine ligand 3 (CCL3), C-C Chemokine ligand 4 (CCL4), Granzyme B (GZMB), Interleukin-1 α (IL-1a), and Matrix metallopeptidase 8 (MMP8). PERSEVERE additionally takes patient age into account.

The PERSEVERE decision tree has 8 terminal nodes. Of these, 3 terminal nodes of the PERSEVERE decision tree are determined to be low risk/low mortality probability (terminal nodes 2, 4, and 7), while 5 terminal nodes of the PERSEVERE decision tree are determined to be intermediate to high risk/high mortality probability (terminal nodes 1, 3, 5, 6, and 8). In some embodiments, a low risk/low mortality probability terminal nodes has a mortality probability between 0.000 and 0.025, while an intermediate to high risk/high mortality probability terminal nodes has a mortality probability greater than 0.025.

In some embodiments of the present disclosure, a patient sample is analyzed for the PERSEVERE serum protein biomarkers IL-8 and HSP70, as well as for the endothelial biomarkers ICAM-1, Thrombomodulin, Angpt-2/Angpt-1, and/or Angpt-2/Tie-2.

The PERSEVERE II model for estimating baseline mortality risk in children with septic shock was previously derived and validated. PERSEVERE II is based on a panel of 5 serum protein biomarkers measured from blood samples obtained during the first 24 hours of a septic shock diagnosis. Of those 5 serum biomarkers, the derived and validated PERSEVERE II model is based on interleukin-8 (IL-8), C-C chemokine ligand 3 (CCL3), and heat shock protein 70 kDa 1B (HSPA1B), as well as platelet count.

The PERSEVERE II decision tree has 5 terminal nodes. Of these, 3 terminal nodes of the PERSEVERE II decision tree are determined to be low risk/low mortality probability (terminal nodes 1, 2, and 4), while 2 terminal nodes of the PERSEVERE II decision tree are determined to be intermediate to high risk/high mortality probability (terminal nodes 3 and 5). In some embodiments, a low risk/low mortality probability terminal nodes has a mortality probability between 0.000 and 0.025, while an intermediate to high risk/high mortality probability terminal nodes has a mortality probability greater than 0.025.

In some embodiments of the present disclosure, a patient sample is analyzed for the PERSEVERE II serum protein biomarkers IL-8, CCL3, and HSPA1B, and platelet count, as well as for the endothelial biomarkers Tie-2, Angpt-2, and sTM.

In some embodiments of the present disclosure, the PERSEVERE and/or PERSEVERE II mortality probability stratification can be used in combination with biomarker-based MODS and/or mortality risk stratification as described herein. In some embodiments, the biomarker-based MODS and/or mortality risk stratification, as described herein, can be used in combination with a patient endotyping strategy and/or Z score determination. In some embodiments, the combination of a biomarker-based MODS and/or mortality risk stratification, with an endotyping strategy and/or Z score determination, can be used to determine an appropriate treatment regimen for a patient. For example, such combinations can be used to identify which patients are more likely to benefit from corticosteroids.

A number of additional models that generate mortality prediction scores based on physiological variables have been developed to date. These can include the PRISM, Pediatric Index of Mortality (PIM), and/pediatric logistic organ dysfunction (PELOD) models, and the like.

Such models can be very effective for estimating population-based outcome risks but are not intended for stratification of individual patients. The methods described herein which allow for stratification of individual patients can be used alone or in combination with one or more existing population-based risk scores.

In some embodiments, the biomarker-based MODS and/or mortality risk stratification described herein can be used with one or more additional population-based risk scores. In some embodiments, the biomarker-based MODS and/or mortality risk stratification described herein can be used in combination with PRISM. In some embodiments, the biomarker-based MODS and/or mortality risk stratification described herein can be used in combination with PIM. In some embodiments, the biomarker-based MODS and/or mortality risk stratification described herein can be used in combination with PELOD. In some embodiments, the biomarker-based MODS and/or mortality risk stratification described herein can be used in combination with a population-based risk score other than PRISM, PIM, and PELOD.

### High Risk Therapies

High risk, invasive therapeutic and support modalities can be used to treat septic shock. The methods described herein which allow for the patient's outcome risk to be determined can help inform clinical decisions regarding the application of high risk therapies to specific pediatric patients, based on the patient's outcome risk.

High risk therapies include, for example, adjuvant hemoperfusion, adjuvant hemoperfusion, extracorporeal hemadsorption, plasma filtration and adsorption therapies, extracorporeal membrane oxygenation/life support, plasmapheresis, pulmonary artery catheterization, high volume continuous hemofiltration, and the like. High risk therapies can also include non-corticosteroid therapies, e.g. alternative therapies and/or high risk therapies. In particular, patients at high risk of MODS and/or mortality risk stratification can be treated with immune enhancing therapies, such as, for example, interleukin-1 receptor antagonist (Anakinra), GMCSF, interleukin-7, anti-PD-1, and the like.

In some embodiments, individualized treatment can be provided to a pediatric patient by selecting a pediatric patient classified as high risk by the methods described herein for one or more high risk therapies. In some embodiments, individualized treatment can be provided to a pediatric patient by excluding a pediatric patient classified as low risk from one or more high risk therapies.

### Computer Implemented System

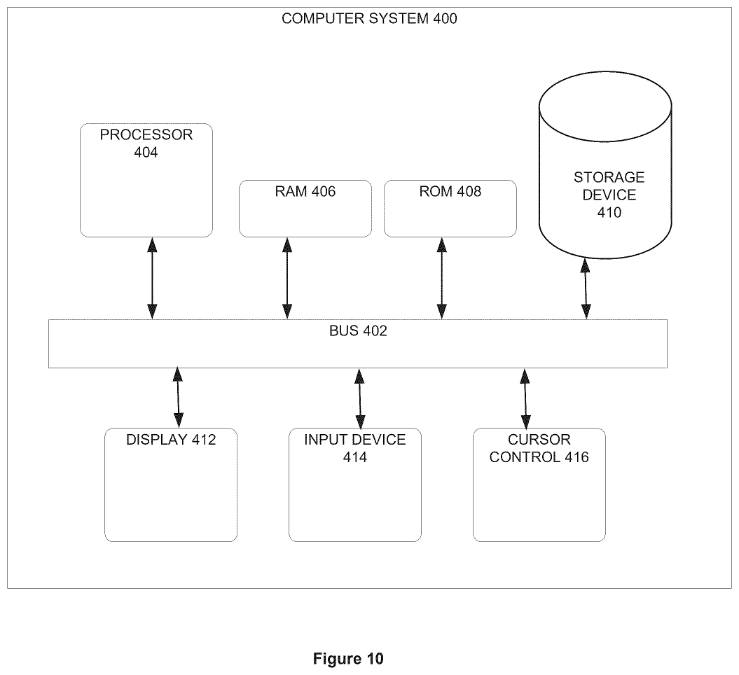

FIG. 10 is a block diagram that illustrates a computer system 400, upon which embodiments of the present teachings may be implemented. Computer system 400 may be an example of one implementation for the computations used in the methods described above and in the examples which follow.

In various embodiments of the present teachings, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT), liquid crystal display (LCD), or light emitting diode (LED) for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

Certain embodiments of the disclosure include using quantification data from a gene-expression analysis and/or from a protein, mRNA, and/or DNA analysis, from a sample of blood, urine, saliva, broncho-alveolar lavage fluid, or the like. Embodiments of the disclosure include not only methods of conducting and interpreting such tests but also include reagents, compositions, kits, tests, arrays, apparatuses, processing devices, assays, and the like, for conducting the tests. The compositions and kits of the present disclosure can include one or more components which enable detection of the biomarkers disclosed herein and combinations thereof and can include, but are not limited to, primers, probes, cDNA, enzymes, covalently attached reporter molecules, and the like.

Diagnostic-testing procedure performance is commonly described by evaluating control groups to obtain four critical test characteristics, namely positive predictive value (PPV), negative predictive value (NPV), sensitivity, and specificity, which provide information regarding the effectiveness of the test. The PPV of a particular diagnostic test represents the proportion of positive tests in subjects with the condition of interest (i.e. proportion of true positives); for tests with a high PPV, a positive test indicates the presence of the condition in question. The NPV of a particular diagnostic test represents the proportion of negative tests in subjects without the condition of interest (i.e. proportion of true negatives); for tests with a high NPV, a negative test indicates the absence of the condition. Sensitivity represents the proportion of subjects with the condition of interest who will have a positive test; for tests with high sensitivity, a positive test indicates the presence of the condition in question. Specificity represents the proportion of subjects without the condition of interest who will have a negative test; for tests with high specificity, a negative test indicates the absence of the condition.

The threshold for the disease state can alternatively be defined as a 1-D quantitative score, or diagnostic cutoff, based upon receiver operating characteristic (ROC) analysis. The quantitative score based upon ROC analysis can be used to determine the specificity and/or the sensitivity of a given diagnosis based upon subjecting a patient to a decision tree described herein in order to predict an outcome for a pediatric patient with septic shock.

The correlations disclosed herein, between pediatric patient septic shock biomarker levels and/or mRNA levels and/or gene expression levels, and/or protein expression levels, provide a basis for conducting a diagnosis of septic shock, or for conducting a stratification of patients with septic shock, or for enhancing the reliability of a diagnosis of septic shock by combining the results of a quantification of a septic shock biomarker with results from other tests or indicia of septic shock, or for determining an appropriate treatment regimen for a pediatric patient with septic shock. For example, the results of a quantification of one biomarker could be combined with the results of a quantification of one or more additional biomarker, protein, cytokine, mRNA, or the like. Thus, even in situations in which a given biomarker correlates only moderately or weakly with septic shock, providing only a relatively small PPV, NPV, specificity, and/or sensitivity, the correlation can be one indicium, combinable with one or more others that, in combination, provide an enhanced clarity and certainty of diagnosis. Accordingly, the methods and materials of the disclosure are expressly contemplated to be used both alone and in combination with other tests and indicia, whether quantitative or qualitative in nature.

Having described various embodiments in detail, it will be apparent that modifications, variations, and equivalent embodiments are possible without departing from the scope of the embodiments defined in the appended claims. Furthermore, it should be appreciated that all examples in the present disclosure are provided as non-limiting examples.

### EXAMPLES

The following non-limiting examples are provided to further illustrate embodiments disclosed herein. It should be appreciated by those of skill in the art that the techniques disclosed in the examples that follow represent approaches that have been found to function well in the practice of the embodiments disclosed herein, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of those embodiments.

### Example 1

### Methods

The methods used in Examples 2-6 are summarized below:

### Study Design and Patient Selection.

The study protocol was approved by Institutional Review Boards of participating institutions [13,15]. Briefly, patients under the age of 18 years were recruited from multiple pediatric ICUs (PICU) across the U.S. between 2003 and 2019. Inclusion criteria were pediatric-specific consensus criteria for septic shock [21] and patients with existing PERSEVERE biomarker data. There were no study related interventions except for blood draws. Clinical and laboratory data were available between day 1 through 7. Patients were followed for 28 days or until death, whichever came first. Organ dysfunctions were determined based on modifications to consensus criteria [21] and are described below.

The primary outcome of interest was a composite that included patients who died before day 7 or those with ≥2 organ dysfunctions on day 7 of septic shock. We chose this composite outcome based on the assumption that 1) non-survivors died due to or with MODS, and that 2) non-survivors or those with persistence of organ dysfunctions on day 7, despite intensive organ support, represent a subset of patients with a yet unknown biological predilection potentially amenable to therapeutic intervention. Accordingly, there is sufficient clinical equipoise within this collective of patients to justify efforts for enrichment in future clinical trials of novel or repurposed sepsis therapeutics. Secondary outcomes were day 7 cardiovascular, respiratory, renal, hepatic, hematologic, and neurologic dysfunction.

### Definitions and Correlation of Organ Dysfunctions.

Organ dysfunctions were determined based on modifications to pediatric consensus criteria [21]. This study accounts for pre-existing conditions and capture acute organ dysfunctions related to index septic shock admission. Accordingly, patients with pre-existing conditions had to meet more stringent criteria for organ dysfunctions to be considered related to sepsis.

Cardiovascular dysfunction: Patients without pre-existing heart disease were considered to have cardiac dysfunction if they had low mean arterial pressure for age, low heart rate for age, requirement of vasoactive support or cardiac arrest. Patients with pre-existing history of congenital heart disease or pulmonary hypertension were only considered to have new cardiovascular dysfunction if they used vasoactive support or had cardiac arrest.

Respiratory dysfunction: Patients without pre-existing lung disease or pulmonary hypertension were considered to have respiratory dysfunction if meeting ≥1 of the following criteria: requiring endo-tracheal intubation for acute respiratory failure, mechanical ventilation for >24 hours, PaO₂/FiO₂<250, PaCO₂>65, PaO₂<40. Patients with pre-existing lung disease or pulmonary hypertension were considered to have respiratory dysfunction if meeting ≥2 of the above criteria.

Renal dysfunction: Patients with pre-existing renal disease were not considered to have acute renal dysfunction. Those without pre-existing renal disease, renal dysfunction was defined as meeting KDIGO criteria [30] stage ≥2 acute kidney injury or use of renal replacement therapy. Baseline creatinine was estimated by modified Schwarz or Pottel method, according to published methods [16].

Hepatic dysfunction: Patients with pre-existing hepatic disease were not considered to have acute hepatic dysfunction. Those without pre-existing disease, hepatic dysfunction was based on meeting at least one of the following 3 criteria: total bilirubin >4 mg/dL, Alanine aminotransferase levels >2 times upper limit for age and sex, and or gastrointestinal bleeding requiring greater than 20 ml/kg of blood transfusion.

Hematologic dysfunction: Patients without pre-existing hematologic disease, cancer, bone marrow transplantation had to meet ≥1 of the following criteria international normalized ratio (INR) >2, platelet count <80,000 per microliter of blood, or evidence of disseminated intravascular coagulopathy (DIC). Those with pre-existing conditions had to meet ≥2 or the above criteria.

Neurologic dysfunction: Patients with pre-existing neurologic disease including those with hypoxic ischemic encephalopathy, cerebral palsy or epilepsy disorders, were not considered to have neurologic dysfunction. Among those without pre-existing conditions, patients had meet ≥1 of the following criteria: Glasgow coma scale <5, fixed dilated pupils, or intracranial pressure >20 millimeters of mercury to be considered to have neurologic dysfunction.

### Persevere Biomarkers.

Concentrations of Interleukin-8 (IL-8), Heat shock protein 70 kDA (HSP70), C-C Chemokine ligand 3 (CCL3), C-C Chemokine ligand 4 (CCL4), Granzyme B (GZMB), Interleukin-1 α (IL-1a), and Matrix metallopeptidase 8 (MMP8) were previously measured in day 1 serum [13,15].

### Endothelial Dysfunction Markers.

Concentrations of Intercellular adhesion molecule-1 (ICAM-1), Thrombomodulin, Angiopoietin-1 (Angpt-1), Angiopoietin-2 (Angpt-2), Tyrosine kinase with immunoglobulin-like loops and epidermal growth factor homology domains-2 (Tie-2), Vascular cell adhesion molecule-1 (VCAM-1), P-selectin, E-selectin, and Platelet and endothelial cell adhesion molecule-1 (PECAM-1) were measured in day 1 serum by Luminex assays (R&D Systems, MN), according to the manufacturer's specifications.

### Serum Biomarker Assays.

The serum biomarkers were measured through Luminex assays (R&D biosystems) using a custom panel for PERSEVERE and a commercially available biomarker panel for the endothelial dysfunction markers. Measurement was according to the manufacturer's instructions (https://www.rndsystems.com/what-luminex-assay#:˜:text=The%20Luminex% C2%AE%20xMAP%C2%AE,spectral%20signature%2C%20o r%20bead%20region).

In summary, samples having 30 microliter or less of serum were collected on day 1 of septic shock and combined with multiple immunobeads specific to the biomarker of interest. Antibodies specific to a desired analyte were coupled to a unique bead region and incubated with the sample. The resultant read out gives out the serum biomarker concentration. Values out of range were omitted from analyses. These studies have log 10 transformed the values for further analyses.

### Statistical Analyses and Modeling.

Statistical analyses were performed with Minitab® software (v21.1, PA). Demographic and clinical data were summarized with percentages or median with interquartile ranges. Differences between groups were determined by Chi squared test for categorical variables and by one-way analysis of variance (ANOVA) for continuous variables. A p-value of 0.05 was used to test statistical significance. Twenty-two predictors including clinical age, serum lactate, PRISM-III score [22], and day 1 vasoactive inotropic score (VIS) [23] and biological variables (ratios of Angpt-2/Tie-2 and Angpt-2/Angpt-1 and Log (10) transformed PERSEVERE and endothelial marker concentrations) were considered. Multiple logistic regression was used to test the association between individual biomarkers and risk of primary outcome of interest after adjusting for age, sex, and PRISM-III score.

A predictive analytics module with automated machine learning tool was used to discover the best model among TreeNet®, Random Forests®, Classification and Regression Tree (CART®), and logistic regression models. TreeNet® consistently provided the least misclassification and was chosen for model derivation. TreeNet® models, which rely on stochastic gradient boosting, consist of several hundred CART® trees with a limited number of terminal nodes. Iterative steps using recursive data sampling are used to grow additional trees to explain residual error from previous trees. While CART® classification only captures interactions of predictor variables in very specific combinations that influence the outcome together, TreeNet® allows for the capture of the overall effect of one predictor variable over another (see https://www.minitab.com/en-us/predictive-analytics/treenet/). Because TreeNet is a blackbox software and does not give a detailed account of the threshold variables, CART models are presented as alternatives.

Models were weighted to ensure equal sample size across classes to overcome unequal distribution of classes of organ dysfunctions in the training dataset. Second order interactions between biological variables were allowed. An event probability threshold of 0.45 was used to optimize model sensitivity. 10-fold cross-validation was used in test sets. Relative variable importance, defined as percent improvement with respect to the top predictor, was used to select variables to develop a simplified model. Test characteristics of risk prediction models including area under the receiver operator characteristic curve (AUROC), positive and negative predictive values and likelihood ratios were determined. Percent of total squared error and % squared error for the top 2-way interactions between biological variables were assessed. Finally, given the black box nature of TreeNet® models, alternative CART® models were presented to promote open science and allow for external validation. Briefly, models were weighted to match sample frequencies and minimum misclassification cost was chosen to select the optimal tree. Class probability method and 10-fold cross validation was used. CART® trees were pruned to ensure that terminal nodes had >5% of patients of the root node.

### Example 2

### Baseline Characteristics were Established

A total of 502 patients with PERSEVERE and endothelial marker data were included in this study. Table 1 shows the demographic data of the cohort by presence of death or day 7 MODS. Over one-third of the cohort (n=173, 34.5%) patients had the primary outcome of interest, including 49 patients who died within the first 7 days of septic shock. Patients with death or day 7 MODS had higher day 1 VIS scores, were more likely to have received steroids, used more organ support, and had fewer ICU free days.

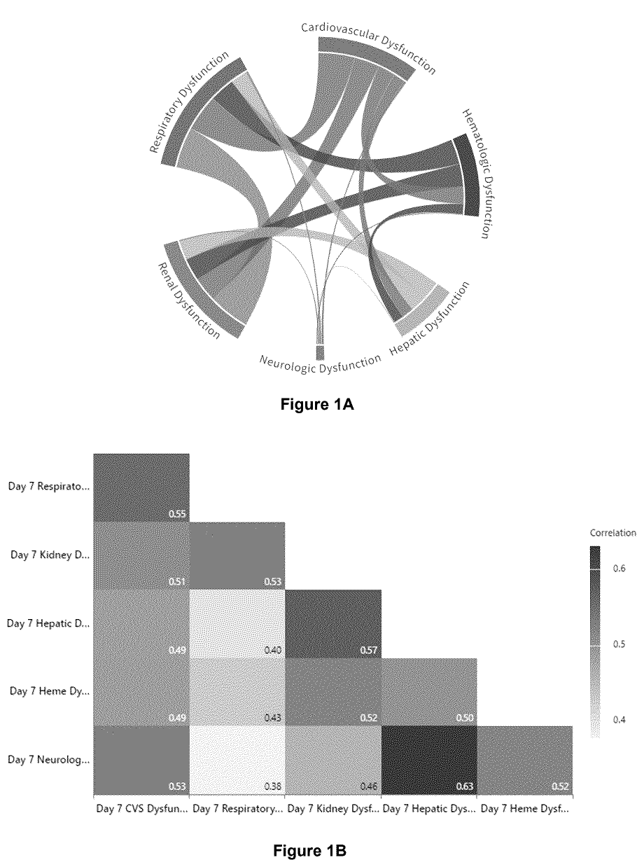

Inter-relationship between individual organ dysfunctions are detailed in FIG. 1. Table 2 shows 28-day mortality among pediatric septic shock patients with and without specific day 7 organ dysfunctions. Concentrations of individual biomarkers by primary outcome and multiple logistic regression analyses to test their association with death or day 7 MODS, adjusted for age, sex, and PRISM III score, are detailed in Table 3 and Table 4.

TABLE 1
Demographic characteristics and clinical outcomes according
to death or day 7 MODS in pediatric septic shock.

| | No death or | Death or | P |
|---|---|---|---|
| Variable | day 7 MODS | day 7 MODS | value |
| n (%) | 329 (65.5%) | 173 (34.5%) | |
| Age | 4.2 (1.5, 8.2) | 2.7 (0.9, 6.6) | 0.007 |
| Sex, M, n(%) | 170 (51.6%) | 92 (53.2%) | 0.748 |
| PRISM III | 10 (6, 15) | 15 (9) | <0.001 |
| Day 1 VIS | 10 (2, 21) | 20 (5, 50) | <0.001 |
| Lactate | 1.2 (0, 2.3) | 1.9 (0.9, 5.1) | <0.001 |
| Positive blood culture | 62 (18.8%) | 44 (25.4%) | 0.086 |
| Positive culture (any) | 174 (52.8%) | 108 (62.4%) | 0.016 |
| Source of infection: | | | 0.030 |
| Pulmonary | 64 (19.4%) | 45 (26.0%) | |
| Extrapulmonary | 110 (33.4%) | 63 (36.4%) | |
| Organism | | | 0.052 |
| Gram positive | 78 (23.7%) | 44 (25.4%) | |
| Gram negative | 60 (18.2%) | 41 (23.6%) | |
| Viral | 22 (6.6%) | 13 (7.5%) | |
| Fungal | 8 (2.4%) | 3 (1.7%) | |
| Organ dysfunctions on day 7 | | | |
| Max. number of organ failures | 0 (0, 1) | 2 (1, 3) | <0.001 |
| Cardiovascular | 9 | 113 | <0.001 |
| Respiratory | 38 | 155 | <0.001 |
| Renal | 14 | 129 | <0.001 |
| Hepatic | 6 | 77 | <0.001 |
| Hematologic | 11 | 96 | <0.001 |
| Neurologic | 0 | 41 | <0.001 |
| Organ support on day 7 | | | |
| Vasoactive support | 11 (3.3%) | 103 (59.5%) | <0.001 |
| Mechanical ventilation | 46 (13.9%) | 158 (91.3%) | <0.001 |
| Renal replacement therapy | 5 (1.5%) | 66 (38.5%) | <0.001 |
| Steroids | 158 (48.1%) | 101 (58.3%) | 0.027 |
| 28-day Mortality | 2 (0.6%) | 61 (35.3%) | <0.001 |
| PICU LOS days | 5 (7) | 12 (13) | <0.001 |
| PICU-free days | 23 (7) | 15 (16) | <0.001 |

TABLE 2
28-day mortality among pediatric septic shock patients
with and without day 7 organ dysfunctions.

| | 28-day mortality | 28-day mortality | |
|---|---|---|---|
| | among patients | among patients | |
| | without day 7 | with day 7 | |
| | organ dysfunction | organ dysfunction | p value |
| Cardiovascular | 9 | 54 | <0.001 |
| Respiratory | 4 | 59 | <0.001 |
| Renal | 5 | 58 | <0.001 |
| Hepatic | 11 | 52 | <0.001 |
| Hematologic | 5 | 58 | <0.001 |
| Neurologic | 24 | 39 | <0.001 |

TABLE 3
Concentrations of serum biomarkers (protein) according to occurrence
of day 7 MODS in children with septic shock. Concentrations
are in nanogram per microliter, and log 10 transformed.

| | | | |
|---|---|---|---|
| Variable | No Day 7 MODS | Day 7 MODS | P value |

PERSEVERE biomarkers:

| | | | |
|---|---|---|---|
| IL-8 (log10) | 2.2 (1.8, 2.6) | 2.9 (2.3, 3.9) | <0.001 |
| HSP70 (log10) | 5.8 (5.5, 6.0) | 6.0 (5.7, 6.4) | <0.001 |
| CXCL3 (log10) | 1.9 (1.6, 2.0) | 2.0 (1.7, 2.3) | <0.001 |
| CXCL4 (log10) | 2.0 (1.8, 2.3) | 2.2 (1.9, 2.6) | <0.001 |
| GZMB (log10) | 1.1 (0.7, 1.5) | 1.3 (0.8, 1.9) | 0.002 |
| IL-1α (log10) | 0.2 (0, 0.8) | 0.4 (0, 0.9) | 0.081 |
| MMP8 (log10) | 4.6 (4.1, 5.0) | 4.6 (4.1, 5.1) | 0.549 |

Endothelial biomarkers:

| | | | |
|---|---|---|---|
| ICAM-1 (log10) | 5.7 (5.6, 5.9) | 5.9 (5.8, 6.1) | <0.001 |
| Thrombomodulin (log10) | 3.8 (3.7, 4.0) | 4.0 (3.8, 4.2) | <0.001 |
| Angpt1 (log10) | 4.3 (4.0, 4.5) | 4.0 (3.7, 4.3) | <0.001 |
| Angpt2 (log10) | 3.8 (3.6, 4.1) | 4.1 (3.9, 4.4) | <0.001 |
| Tie2 (log10) | 4.4 (4.2, 4.5) | 4.3 (4.1, 4.4) | <0.001 |
| Angpt2/Angpt1 | 0.4 (0.2, 1.0) | 1.4 (0.5, 3.5) | <0.001 |
| Angpt2/Tie2 | 0.3 (0.2, 0.5) | 0.8 (0.4, 1.4) | <0.001 |
| VCAM-1 (log10) | 6.4 (6.2, 6.7) | 6.6 (6.3, 6.7) | 0.001 |
| P-selectin (log10) | 4.8 (4.6, 5.0) | 4.7 (4.6, 4.9) | 0.002 |
| E-selectin (log10) | 4.9 (4.7, 5.1) | 4.9 (4.6, 5.1) | 0.374 |
| PECAM-1 (log10) | 4.3 (4.2, 4.5) | 4.3 (4.3, 4.5) | 0.607 |

TABLE 4
Multivariable logistic regression testing for the association
between individual clinical and biological variables and
death or day 7 MODS among children with septic shock.

| | | |
|---|---|---|
| Variable | Adjusted OR, 95% CI | P value |

Clinical variables:

| | | |
|---|---|---|
| Day 1 VIS | 1.00 (1.00-1.01) | 0.025 |
| Lactate | 1.14 (1.07-1.22) | <0.001 |

PERSEVERE biomarkers:

| | | |
|---|---|---|
| IL-8 (log10) | 2.70 (2.04-3.58) | <0.001 |
| HSP70 (log10) | 2.35 (1.60-3.45) | <0.001 |
| CXCL3 (log10) | 2.29 (1.41-3.69) | <0.001 |
| CXCL4 (log10) | 1.63 (1.06-2.51) | 0.02 |
| GZMB (log10) | 1.40 (1.08-1.82) | 0.01 |
| IL-1α | 1.21 (0.90-1.62) | 0.20 |
| MMP8 | 1.02 (0.80-1.32) | 0.823 |

Endothelial biomarkers:

| | | |
|---|---|---|
| ICAM-1 (log10) | 23.29 (8.06-67.31) | <0.001 |
| Thrombomodulin (log10) | 19.73 (7.73-50.36) | <0.001 |
| Angpt1 (log10) | 0.31 (0.20-0.51) | <0.001 |
| Angpt2 (log10) | 6.84 (3.69-12.67) | <0.001 |
| Tie2 (log10) | 0.19 (0.07-0.51) | <0.001 |
| Angpt2/Angpt1 | 1.16 (1.07-1.25) | <0.001 |
| Angpt2/Tie2 | 3.13 (2.15-4.54) | <0.001 |
| VCAM-1 (log10) | 2.37 (1.21-4.62) | 0.011 |
| P-selectin (log10) | 0.28 (0.12-0.66) | 0.004 |
| E-selectin (log10) | 0.79 (0.44-1.41) | 0.422 |
| PECAM-1 (log10) | 0.92 (0.37-2.28) | 0.864 |

Adjusted for age, sex, and PRISM III
*

### Example 3

### The Risk of Death or Persistent Organ Dysfunction on Day 7 of Septic Shock was Estimated

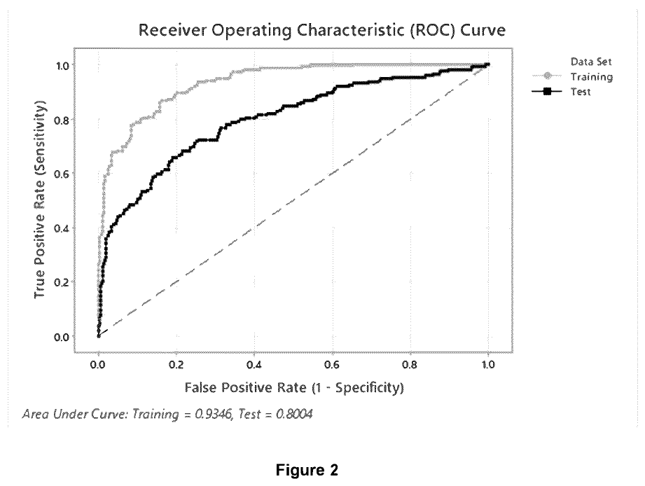

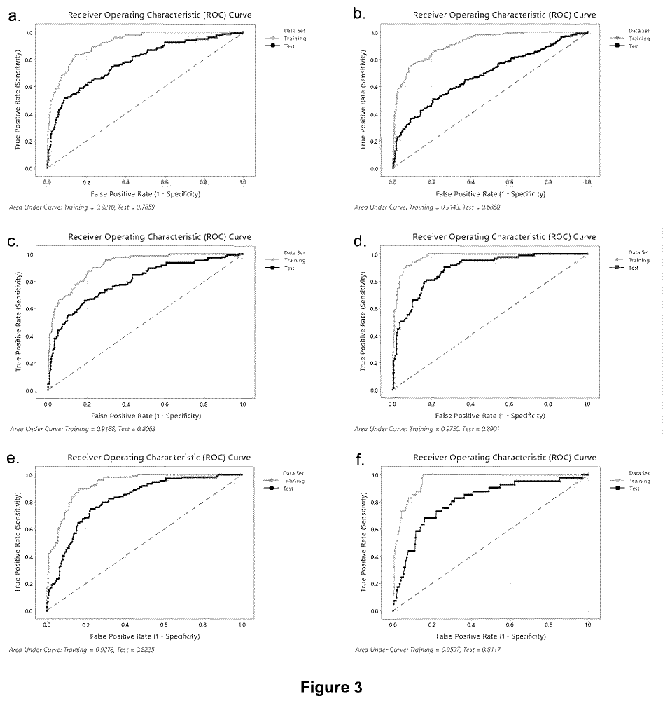

All 22 predictor variables were deemed important in the TreeNet® classification model. 300 trees were grown and 220 was considered as the optimal number of trees. Table 5 shows test characteristics of the newly derived PERSEVEREnce model to estimate risk of death or day 7 MODS. The area under the receiver operator characteristic curve (AUROC) of the training set was 0.93 (95% CI 0.91-0.95) and 0.80 (95% CI: 0.76-0.84) upon 10-fold cross validation as shown in FIG. 2. In comparison, summary AUROCs for clinical, PERSEVERE, and endothelial markers considered separately were 0.69 (95% CI: 0.64-0.74), 0.73 (95% CI: 0.68-0.78) and 0.75 (95% CI: 0.71-0.79) respectively. The weighted misclassification rate of the PERSEVEREnce model was 0.16 and 0.27 and negative predictive values of 92.1% (95% CI: 88.3-94.9) and 83.7% (95% CI: 78.8-87.6) in training and test sets respectively. FIG. 2 depicts the area under the receiver operating characteristic (AUROC) curve for the 22 variable TreeNet® PERSEVEREnce model to estimate risk of persistent day 7 MODS in children with septic shock; FIG. 3 depicts the area under the receiver operating characteristic (AUROC) curve for the 22 variable TreeNet® PERSEVEREnce models to estimate risk of persistent a) cardiovascular, b) respiratory, c) renal, d), hepatic, e) hematologic, and f) neurologic dysfunction on day 7 of pediatric septic shock.

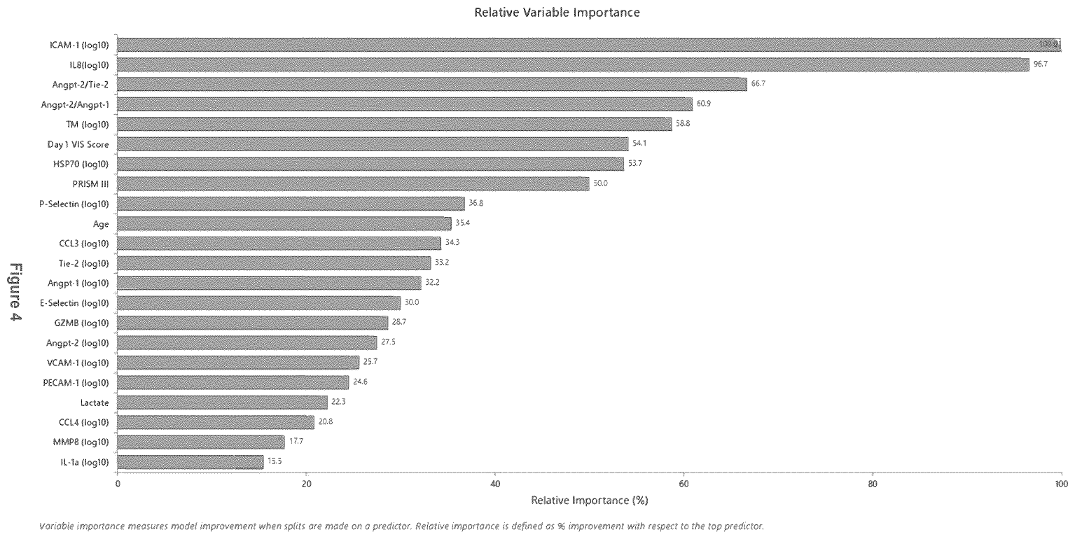

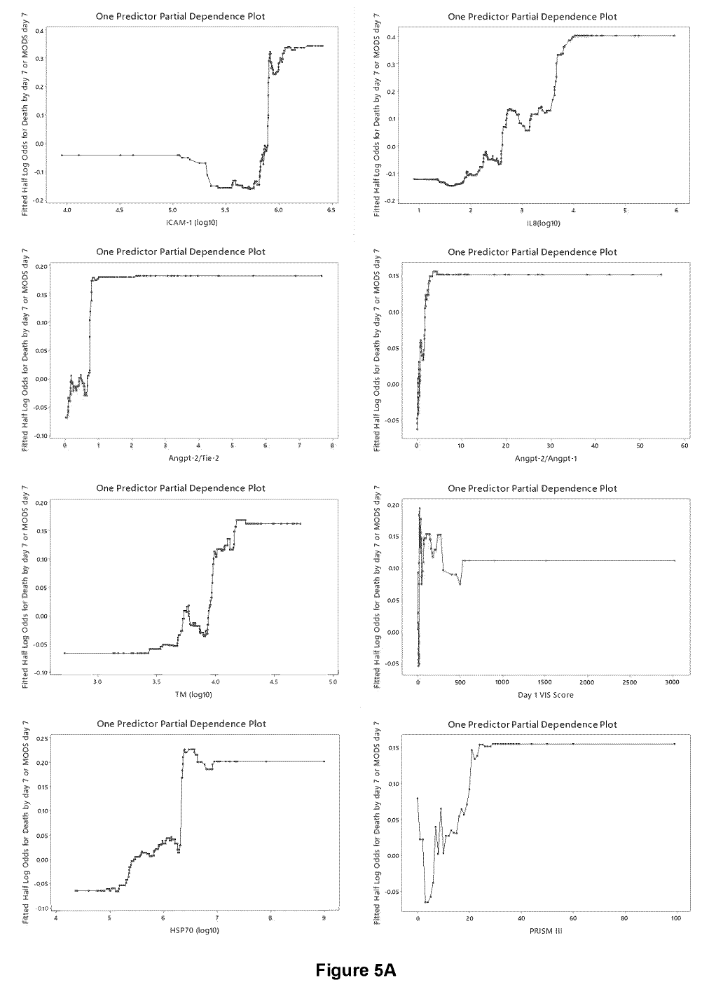

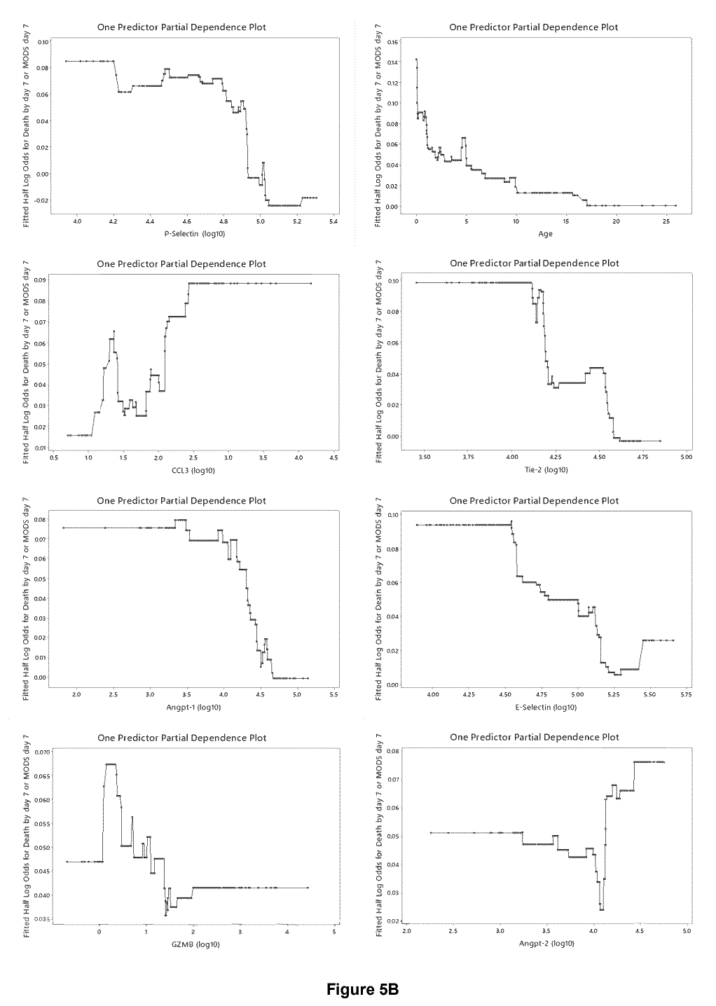

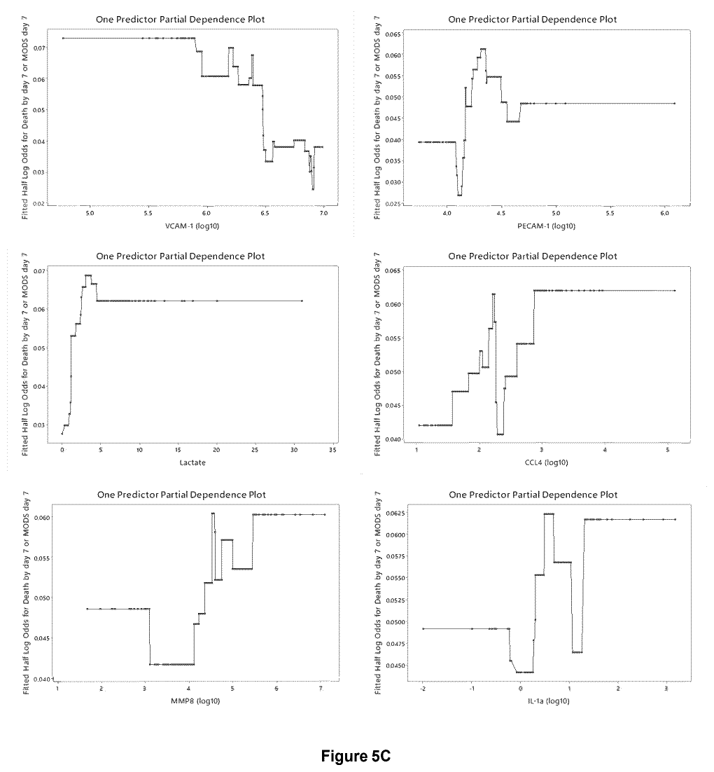

Relative variable importance of predictor variables is shown in FIG. 4. Partial dependence plots of predictor variables are shown in FIG. 5.

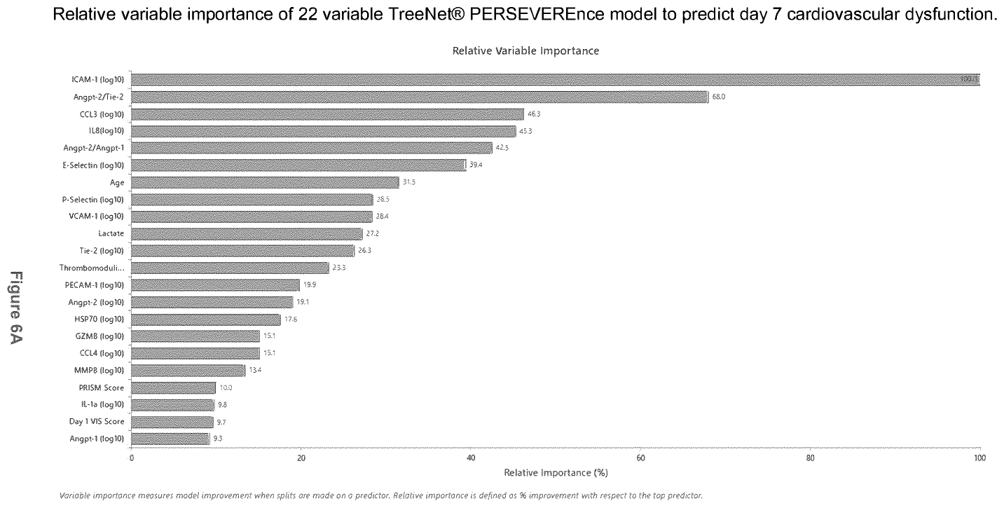

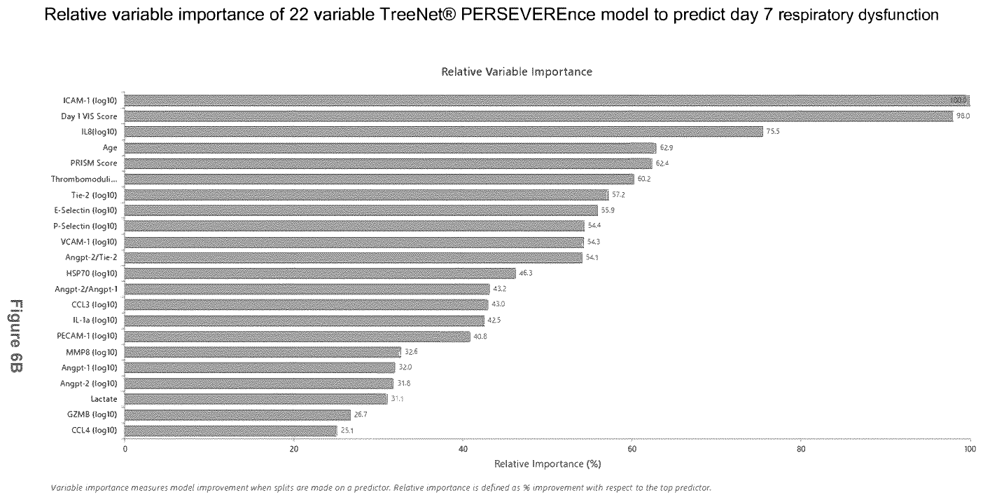

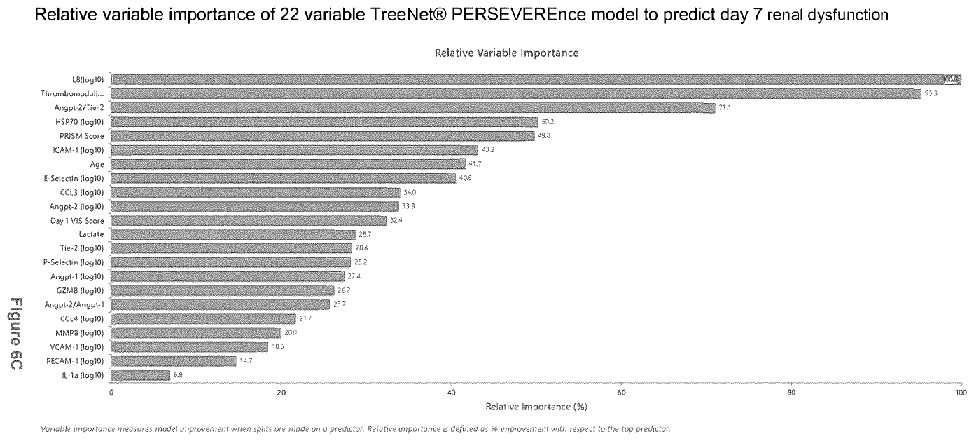

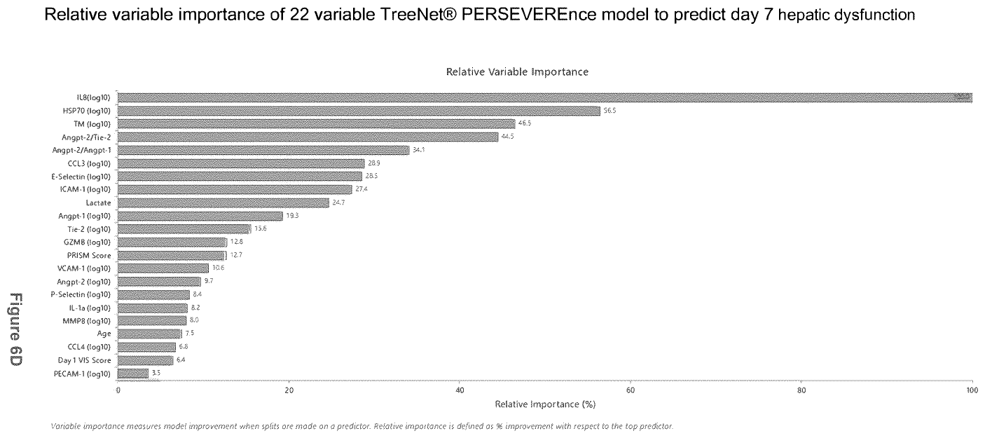

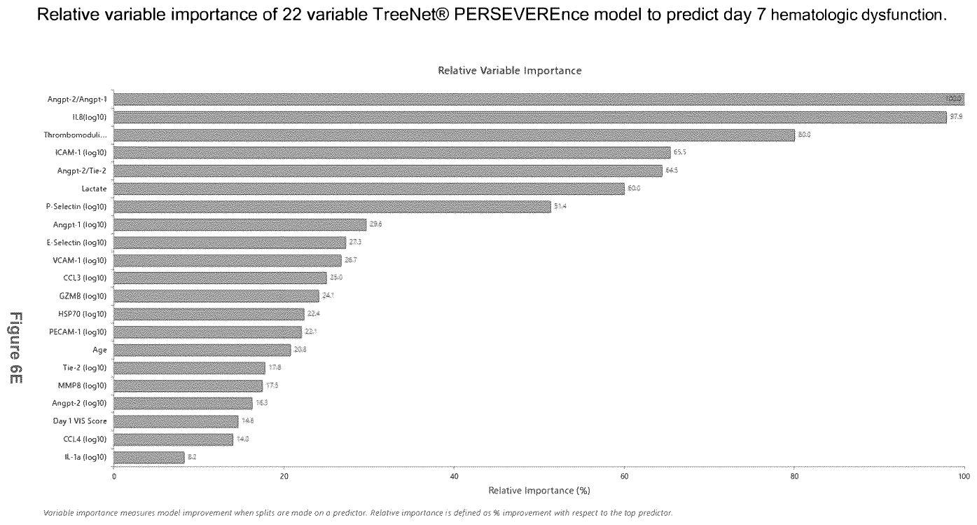

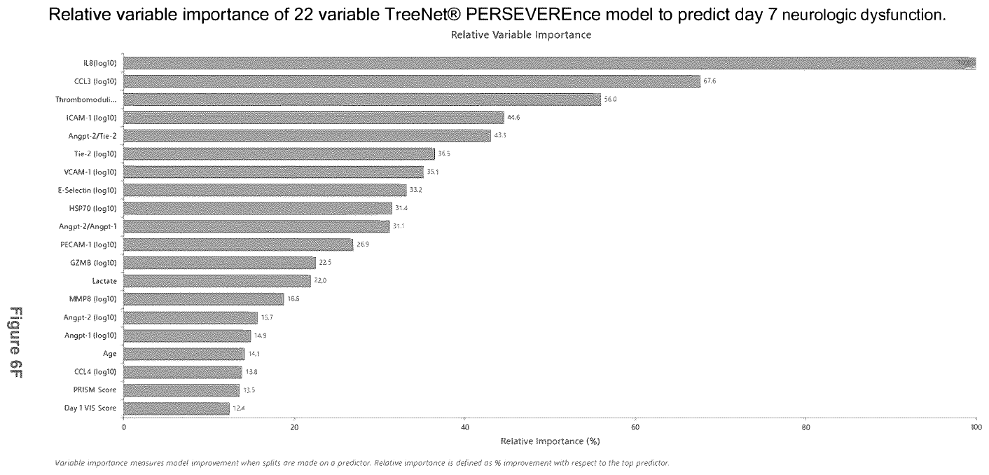

To test whether early deaths due to septic shock (≤48 hours) skewed model performance, a sensitivity analysis was conducted with exclusion of these patients (n=27); model performance was unchanged. The test characteristics of models to estimate risk of individual organ dysfunctions are shown in Table 6. The AUROCs of PERSEVEREnce risk models to predict cardiovascular, respiratory, renal, hepatic, hematologic, and neurologic dysfunctions on day 7 of septic shock ranged from 0.91 to 0.96 and 0.68 to 0.89 in test and training sets respectively and are shown in FIG. 3. The relative importance of predictor variables varied by organ dysfunction, as shown in FIG. 6.

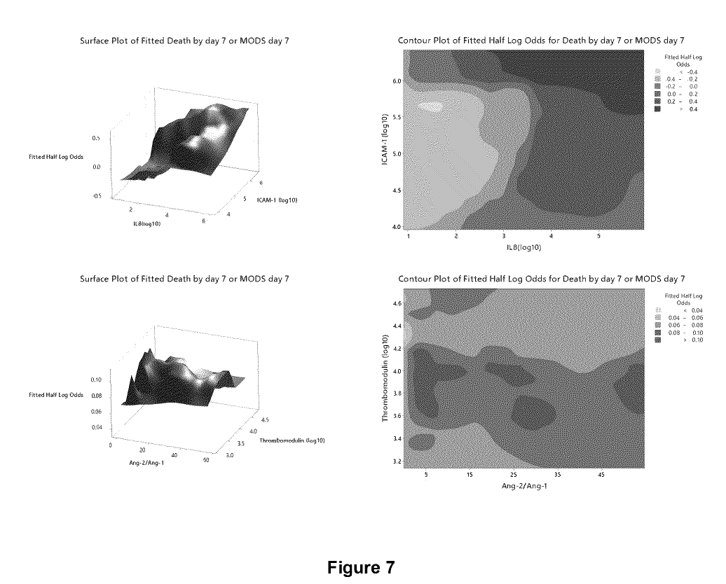

Interaction between PERSEVERE and endothelial markers consistently contributed to the prognostic capabilities of the PERSEVEREnce model to determine risk of death or day 7 MODS. Interaction between ICAM-1 with IL-8 and Thrombomodulin with Angpt-2/Angpt-1 accounted for 7.5 and 2.5% of total squared error and 13.2 and 10.3% of squared error respectively when estimating risk of death or day 7 MODS. FIG. 7 shows surface and contour plots of fitted half log odds of death or day 7 MODS explained by the 2-way interaction of these variables. Table 7 shows the top 2-way interactions between biological variables in organ-specific PERSEVEREnce risk models. Once again, the top interacting variables and the contribution of their interactions to the predictive capabilities of the models varied by organ dysfunction.

TABLE 5
Test characteristics of 22 variable TreeNet ®
PERSEVEREnce model to estimate risk of death or day
7 MODS in children with septic shock.

| | Training Set | Test Set |
|---|---|---|
| AUROC | 0.93 (0.91-0.95) | 0.80 (0.76-0.84) |
| Weighted misclassification rate | 0.16 | 0.26 |
| True positive, n | 150 | 125 |
| False negative, n | 23 | 48 |
| False positive, n | 58 | 83 |
| True negative, n | 271 | 246 |
| Sensitivity % | 86.7 (80.5, 91.2) | 72.2 (64.8, 78.6) |
| Specificity % | 82.3 (77.7, 86.2) | 74.8 (69.7, 79.3) |
| Positive predictive value % | 72.1 (65.4, 77.9) | 60.1 (53.1, 66.7) |
| Negative predictive value % | 92.1 (88.3, 94.9) | 83.7 (78.8, 87.6) |
| Positive likelihood ratio | 4.9 (3.9, 6.3) | 2.9 (2.3, 3.5) |
| Negative likelihood ratio | 0.2 (0.1, 0.3) | 0.4 (0.3, 0.5) |

TABLE 6
Performance of 22 variable organ specific TreeNet ® PERSEVEREnce
risk models.

| | |
|---|---|
| Training Set | Test Set |

Day 7 Cardiovascular Dysfunction

| | | |
|---|---|---|
| AUROC | 0.92 (0.89, 0.94) | 0.79 (0.73, 0.84) |
| Weighted misclassification rate | 0.17 | 0.29 |
| True positive, n | 103 | 80 |
| False negative, n | 19 | 42 |
| False positive, n | 71 | 91 |
| True negative, n | 309 | 289 |
| Sensitivity % | 84.4 (76.4, 90.1) | 65.6 (56.3, 73.7) |
| Specificity % | 81.3 (76.9, 85.0) | 76.1 (71.3, 80.2) |
| Positive predictive value % | 59.1 (51.4, 66.4) | 46.7 (39.1, 54.5) |
| Negative predictive value % | 94.2 (90.0, 96.3) | 87.3 (83.1, 90.6) |

Day 7 Respiratory Dysfunction

| | | |
|---|---|---|
| AUROC | 0.91 (0.89, 0.94) | 0.68 (0.63, 0.73) |
| Weighted misclassification rate | 0.17 | 0.36 |
| True positive, n | 167 | 126 |
| False negative, n | 26 | 67 |
| False positive, n | 68 | 116 |
| True negative, n | 241 | 193 |
| Sensitivity % | 71.1 (64.7, 76.7) | 65.2 (58.1, 71.9) |
| Specificity % | 77.9 (72.8, 82.4) | 62.4 (56.8, 67.8) |
| Positive predictive value % | 71.1 (64.7, 76.6) | 52.1 (45.5, 58.4) |
| Negative predictive value % | 78.0 (72.9, 82.4) | 74.2 (68.3, 79.3) |

Day 7 Renal Dysfunction

| | | |
|---|---|---|
| AUROC | 0.91 (0.89, 0.94) | 0.80(0.76, 0.85) |
| Weighted misclassification rate | 0.17 | 0.27 |
| True positive, n | 123 | 103 |
| False negative, n | 20 | 40 |
| False positive, n | 73 | 96 |
| True negative, n | 286 | 263 |
| Sensitivity % | 86.1 (78.9, 91.1) | 72.1 (63.8, 77.7) |
| Specificity % | 79.7 (75.1, 83.6) | 73.2 (68.3, 77.7) |
| Positive predictive value % | 62.7 (55.5, 69.4) | 51.7 (44.6, 58.8) |
| Negative predictive value % | 93.4 (89.9, 95.9) | 86.7 (82.3, 90.2) |

Day 7 Hepatic Dysfunction

| | | |
|---|---|---|
| AUROC | 0.97 (0.96, 0.99) | 0.89 (0.85, 0.92) |
| Weighted misclassification rate | 0.08 | 0.18 |
| True positive, n | 79 | 66 |
| False negative, n | 4 | 17 |
| False positive, n | 53 | 68 |
| True negative, n | 366 | 351 |
| Sensitivity % | 95.1 (87.5, 98.4) | 79.5 (68.9, 87.2) |
| Specificity % | 87.3 (83.6, 90.3) | 83.7 (79.8, 87.1) |
| Positive predictive value % | 59.8 (50.9, 68.1) | 49.2 (40.5, 57.9) |
| Negative predictive value % | 98.9 (97.1, 99.6) | 95.3 (92.5, 97.2) |

Day 7 Hematologic Dysfunction

| | | |
|---|---|---|
| AUROC | 0.92 (0.90, 0.95) | 0.82 (0.78, 0.86) |
| Weighted misclassification rate | 0.14 | 0.24 |
| True positive, n | 96 | 79 |
| False negative, n | 11 | 28 |
| False positive, n | 75 | 87 |
| True negative, n | 320 | 308 |
| Sensitivity % | 89.7 (81.9, 94.5) | 73.8 (64.2, 81.6) |
| Specificity % | 81.1 (76.7, 84.7) | 77.9 (73.4, 81.9) |
| Positive predictive value % | 56.1 (48.3, 63.6) | 47.5 (39.8, 55.4) |
| Negative predictive value % | 96.6 (93.9, 98.2) | 91.6 (88.1, 94.2) |

Day 7 Neurologic Dysfunction

| | | |
|---|---|---|
| AUROC | 0.96 (0.94, 0.98) | 0.81 (0.74, 0.88) |
| Weighted misclassification rate | 0.15 | 0.25 |
| True positive, n | 38 | 28 |
| False negative, n | 3 | 13 |
| False positive, n | 68 | 73 |
| True negative, n | 393 | 388 |
| Sensitivity % | 92.7 (78.9, 98.1) | 68.2 (51.7, 81.4) |
| Specificity % | 85.2 (81.6, 88.2) | 84.1 (80.4, 87.3) |
| Positive predictive value % | 35.8 (26.9, 45.8) | 27.7 (19.5, 37.6) |
| Negative predictive value % | 99.2 (97.6, 99.8) | 96.7 (94.3, 98.1) |

TABLE 7
Top 2-way interactions between predictors in 22 variable
TreeNet ® PERSEVEREnce risk models.

| | % of total | % of | | |
|---|---|---|---|---|
| | squared | squared | | |
| Outcome | error ** | error *** | Predictor 1 | Predictor 2 |
| Day 7 CVS | 7.5 | 21.1 | IL-8 (Log10) | Angpt-2/Tie-2 |
| Dysfunction | 4.1 | 20.1 | CCL3 (log10) | Angpt-2/Angpt-1 |
| Day 7 Resp | 2.4 | 12.1 | HSP70 (log10) | IL-8 (Log10) |
| Dysfunction | 1.8 | 10.3 | VCAM-1 | Angpt-2/Tie-2 |
| Day 7 Renal | 11.3 | 17.5 | IL-8 (Log10) | Thrombomodulin (log10) |
| Dysfunction | 6.5 | 12.5 | IL-8 (Log10) | Angpt-2/Tie-2 |
| Day 7 Hepatic | 8.5 | 11.6 | IL-8 (Log10) | Angpt-2/Angpt-1 |
| Dysfunction | 7.8 | 10.6 | IL-8 (Log10) | Thrombomodulin (log10) |
| Day 7 Hematologic | 8.3 | 13.7 | IL-8 (Log10) | Angpt-2/Angpt-1 |
| Dysfunction | 5.6 | 13.3 | Thrombomodulin (log10) | Angpt-2/Angpt-1 |
| Day 7 Neurologic | 26.9 | 38.8 | IL-8 (Log10) | Thrombomodulin (log10) |
| Dysfunction | 6.6 | 18.6 | CCL3 (log10) | Angpt-2/Tie-2 |

** Percent of total variation in model that can be attributed to the two predictors including main effects and 2-way interaction.
*** Percent of variation in main and interaction effects of 2 predictor variables that can be
attributed to the 2-way interaction effect.

### Example 4

### Simplified PERSEVEREnce Risk Models were Validated

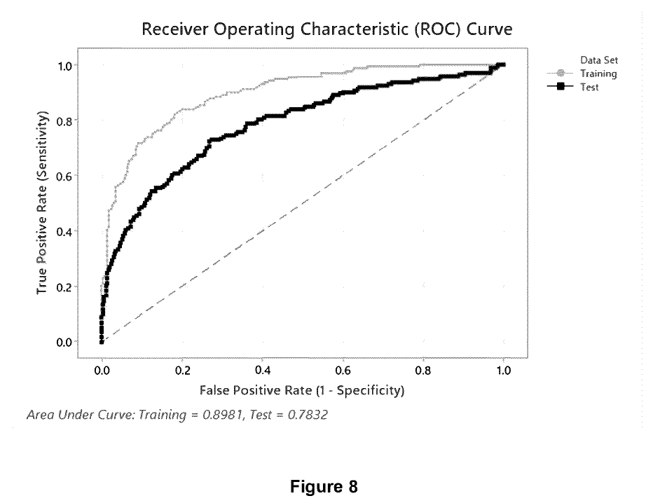

The top 6 biological variables including two PERSEVERE biomarkers IL-8 and HSP70, and four endothelial dysfunction markers ICAM-1, Thrombomodulin, Angpt-2/Angpt-1, Angpt-2/Tie-2, selected based on a relative variable importance threshold of >50% of top predictor, were used to develop simplified TreeNet® models to estimate risk of death or persistent organ dysfunction on day 7 of septic shock. When estimating risk of death or day 7 MODS, 206 trees were considered as the optimal number of trees. The simplified PERSEVEREnce biomarker model had an AUROC of 0.89 (0.87-0.92) and 0.78 (0.75-0.83) as shown in FIG. 8. The weighted misclassification rate was 0.18 and 0.27 in training and test sets respectively. The remaining test characteristics are presented in Table 8. The AUROCs of the organ specific PERSEVEREnce models to predict cardiovascular, respiratory, renal, hepatic, hematologic, and neurologic dysfunctions on day 7 of septic shock ranged between 0.84-0.97 and 0.66-0.88 in training and test sets respectively. Corresponding test characteristics are presented in Table 9. The top 2-way interaction between the 6 variables and their contribution to each risk prediction model are shown in Table 10.

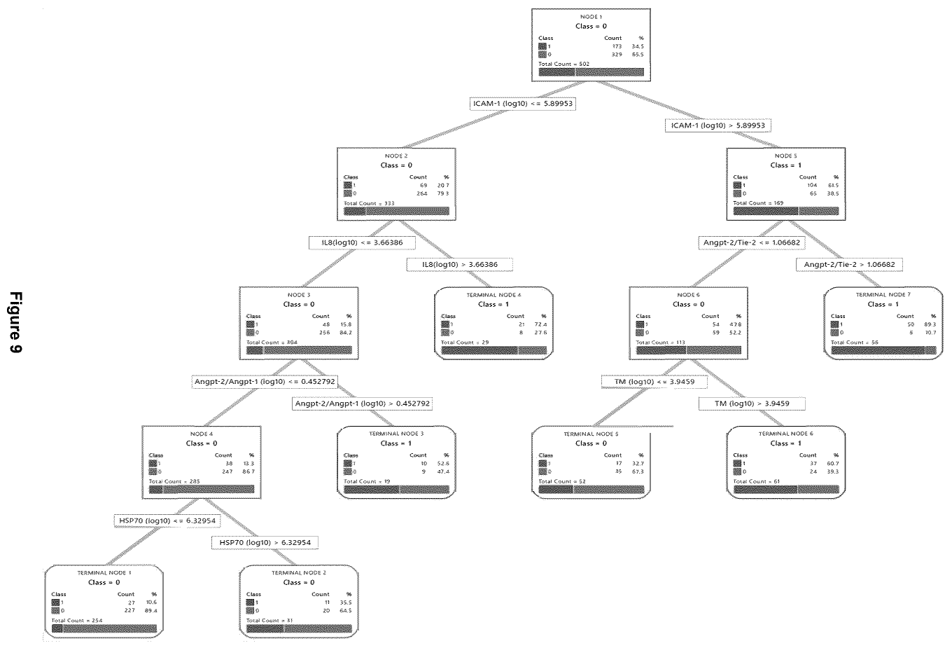

FIG. 9 shows a 7-terminal node PERSEVEREnce CART® model to estimate risk of death or day 7 MODS. Consistent with TreeNet® models, ICAM-1, IL-8, Angpt-2/Angpt-1, and Thrombomodulin influenced classification of patients and featured high in the tree. There were 1 low-risk terminal nodes (TN-1, with 10.6% risk of death or day 7 MODS), 3 intermediate risk terminal nodes (TN-2, 3, and 5, with 32.7-52.6% risk of death or day 7 MODS), and 3 high-risk terminal nodes (TN-4, 6, 7, with 60.7-89.3% risk of death or day 7 MODS). Of note, CART® models had significantly higher misclassification, namely 0.65 and 0.84 in training and test sets, in comparison with TreeNet® models. The AUROC for CART® PERSEVEREnce model was 0.83 and 0.73 on 10-fold cross-validation.

The PERSEVEREnce CART model was subsequently validated in 268 subjects. As shown in Tables 11-13, this model works well to identify patients at risk of death or MODS on day 7.

TABLE 8
Test characteristics of the 6 variable TreeNet ® PERSEVEREnce model
to estimate risk of day 7 MODS in children with septic shock.

| | Training Set | Test Set |
|---|---|---|
| AUROC | 0.89 (0.87-0.93) | 0.78 (0.74-0.83) |
| Weighted misclassification rate | 0.18 | 0.27 |
| True positive, n | 145 | 126 |
| False negative, n | 28 | 47 |
| False positive, n | 65 | 88 |
| True negative, n | 264 | 288 |
| Sensitivity % | 83.8 (77.2, 88.8) | 72.8 (65.4, 79.1) |
| Specificity % | 80.2 (75.4, 84.3) | 76.5 (71.9, 80.7) |
| Positive predictive value % | 69.1 (62.2, 75.1) | 58.8 (51.9, 65.4) |
| Negative predictive value % | 90.4 (86.3, 93.4) | 85.9 (81.6, 89.4) |
| Positive likelihood ratio | 4.2 (3.4, 5.3) | 3.1 (2.5, 3.8) |
| Negative likelihood ratio | 0.1 (0.1, 0.2) | 0.2 (0.1, 0.3) |

TABLE 9
Performance of 6 variable organ specific TreeNet ® PERSEVEREnce
risk models.

| | |
|---|---|
| Training Set | Test Set |

Day 7 Cardiovascular Dysfunction

| | | |
|---|---|---|
| AUROC | 0.87 (0.84, 0.91) | 0.77 (0.72, 0.83) |
| Weighted misclassification rate | 0.22 | 0.29 |
| True positive, n | 108 | 97 |
| False negative, n | 14 | 25 |
| False positive, n | 124 | 139 |
| True negative, n | 256 | 241 |
| Sensitivity % | 88.5 (81.1, 93.3) | 79.5 (71.1, 86.1) |
| Specificity % | 67.3 (62.3, 71.1) | 63.4 (58.3, 68.2) |
| Positive predictive value % | 46.5 (40.1, 53.1) | 41.1 (34.8, 47.6) |
| Negative predictive value % | 94.8 (91.3, 97.1) | 90.6 (86.2, 93.7) |

Day 7 Respiratory Dysfunction

| | | |
|---|---|---|
| AUROC | 0.84 (0.91, 0.87) | 0.66 (0.61, 0.71) |
| Weighted misclassification rate | 0.24 | 0.38 |
| True positive, n | 149 | 120 |
| False negative, n | 44 | 73 |
| False positive, n | 80 | 120 |
| True negative, n | 229 | 189 |
| Sensitivity % | 77.2 (70.5, 82.7) | 62.1 (54.9, 68.9) |
| Specificity % | 74.1 (68.7, 78.8) | 61.1 (55.4, 66.5) |
| Positive predictive value % | 65.0 (58.4, 71.1) | 50.0 (43.5, 56.4) |
| Negative predictive value % | 83.8 (78.8, 87.9) | 72.1 (66.3, 77.3) |

Day 7 Renal Dysfunction

| | | |
|---|---|---|
| AUROC | 0.90 (0.87, 0.93) | 0.79 (0.75, 0.79) |
| Weighted misclassification rate | 0.19 | 0.27 |
| True positive, n | 120 | 102 |
| False negative, n | 23 | 41 |
| False positive, n | 76 | 87 |
| True negative, n | 283 | 272 |
| Sensitivity % | 83.9 (76.6, 89.3) | 71.3 (63.0, 78.4) |
| Specificity % | 78.8 (74.1, 82.8) | 75.7 (70.9, 80.0) |
| Positive predictive value % | 61.2 (54.0, 68.0) | 53.9 (46.5, 61.1) |
| Negative predictive value % | 92.4 (88.7, 95.1) | 86.9 (82.5, 90.3) |

Day 7 Hepatic Dysfunction

| | | |
|---|---|---|
| AUROC | 0.97 (0.96, 0.98) | 0.88 (0.85, 0.89) |
| Weighted misclassification rate | 0.08 | 0.20 |
| True positive, n | 80 | 64 |
| False negative, n | 3 | 19 |
| False positive, n | 55 | 64 |
| True negative, n | 364 | 355 |
| Sensitivity % | 96.3 (89.1, 99.1) | 77.1 (66.3, 85.3) |
| Specificity % | 86.8 (83.1, 89.8) | 84.7 (80.8, 88.0) |
| Positive predictive value % | 59.2 (50.4, 67.5) | 50.0 (41.0, 58.9) |
| Negative predictive value % | 99.2 (97.4, 99.7) | 94.9 (92.1, 96.8) |

Day 7 Hematologic Dysfunction

| | | |
|---|---|---|
| AUROC | 0.93 (0.91, 0.96) | 0.82 (0.77, 0.86) |
| Weighted misclassification rate | 0.14 | 0.25 |
| True positive, n | 95 | 75 |
| False negative, n | 12 | 32 |
| False positive, n | 66 | 81 |
| True negative, n | 329 | 314 |
| Sensitivity % | 88.7 (80.8, 93.8) | 70.1 (60.3, 78.3) |
| Specificity % | 83.3 (79.1, 86.7) | 79.4 (75.1, 83.2) |
| Positive predictive value % | 59.0 (50.9, 66.6) | 48.1 (40.1, 56.1) |
| Negative predictive value % | 96.4 (93.7, 98.1) | 90.7 (87.1, 93.5) |

Day 7 Neurologic Dysfunction

| | | |
|---|---|---|
| AUROC | 0.92 (0.89, 0.95) | 0.81 (0.74, 0.88) |
| Weighted misclassification rate | 0.13 | 0.22 |
| True positive, n | 38 | 35 |
| False negative, n | 3 | 6 |
| False positive, n | 90 | 134 |
| True negative, n | 371 | 327 |
| Sensitivity % | 92.7 (78.9, 98.1) | 85.4 (70.1, 93.9) |
| Specificity % | 80.4 (76.4, 83.9) | 70.9 (66.5, 75.0) |
| Positive predictive value % | 29.7 (22.1, 38.5) | 20.7 (15.0, 27.7) |
| Negative predictive value % | 99.1, 97.4, 99.7) | 98.1 (95.9, 99.2) |

TABLE 10
Top 2-way interactions between predictors in 6 variable
TreeNet ® PERSEVEREnce risk models.

| | % of total | % of | | |
|---|---|---|---|---|
| | squared | squared | | |
| Outcome | error ** | error *** | Predictor 1 | Predictor 2 |
| Day 7 CVS | 12.3 | 26.5 | IL-8 (Log10) | Angpt-2/Tie-2 |
| Dysfunction | 12.3 | 13.9 | ICAM-1 | Angpt-2/Tie-2 |
| Day 7 Resp | 8.3 | 19.6 | HSP70 (log10) | ICAM-1 (log10) |
| Dysfunction | 8.2 | 12.2 | VCAM-1 | Angpt-2/Tie-2 |
| Day 7 Renal | 11.6 | 16.3 | IL-8 (Log10) | Thrombomodulin (log10) |
| Dysfunction | 9.7 | 15.6 | IL-8 (Log10) | Angpt-2/Tie-2 |
| Day 7 Hepatic | 10.4 | 13.9 | IL-8 (Log10) | IL-8 (Log10) |
| Dysfunction | 10.3 | 12.4 | HSP70 (log10) | Thrombomodulin (log10) |
| Day 7 Hematologic | 12.3 | 18.5 | IL-8 (Log10) | Angpt-2/Angpt-1 |
| Dysfunction | 6.4 | 11.1 | Thrombomodulin (log10) | Angpt-2/Angpt-1 |
| Day 7 Neurologic | 37.8 | 42.2 | IL-8 (Log10) | Thrombomodulin (log10) |
| Dysfunction | 11.9 | 14.1 | IL-8 (Log10) | Angpt-2/Tie-2 |

** Percent of total variation in model that can be attributed to the two predictors including main effects and 2-way interaction.
*** Percent of variation in main and interaction effects of 2 predictor variables that can be attributed to the 2-way interaction effect.

TABLE 11
Comparison of predicted PERSEVERENCE CART model
and actual presence of death by or day 7 MODS
in the validation cohort (n = 268).

| Complicated course | Yes (n = 83) | No (n = 185) | P value |
|---|---|---|---|
| HIGH risk | 36 (46.8%) | 41 (53.2%) | <0.001 |
| INTERMEDIATE risk | 42 (32.1% | 89 (67.9%) | |
| LOW risk | 5 (8.3%) | 55 (91.7%) | |

TABLE 12
Presence of complicated course based on PERSEVEREnce
terminal nodes in the validation cohort.

| TN | PREDICTED Class | Yes (n = 83) | No (n = 185) | P value |
|---|---|---|---|---|
| TN1 | LOW | 5 (8.3%) | 55 (91.7%) | <0.001 |
| TN2 | INTERMEDIATE | 5 (50.0%) | 5 (50.0%) | |
| TN3 | INTERMEDIATE | 35 (31.8%) | 75 (68.2%) | |
| TN4 | HIGH | 6 (54.5%) | 5 (45.5%) | |
| TN5 | INTERMEDIATE | 2 (18.2%) | 9 (81.8%) | |
| TN6 | HIGH | 8 (50.0%) | 8 (50.0%) | |
| TN7 | HIGH | 22 (44.0% | 28 (56.0%) | |

TABLE 13
Performance of PERSEVEREnce-CART MODS probability
to estimate risk of complicated course and 28-
day mortality using logistic regression models.

| | | |
|---|---|---|
| Variable | Odds ratio | P value |

Complicated course
Univariate model

| | | |
|---|---|---|
| PERSEVERENCE MODS prob. | 11.3 (95% CI: 3.7, 34.1) | <0.001 |

Multivariate model

| | | |
|---|---|---|
| Age | 0.93 (95% CI: 0.87, 0.98 | 0.012 |
| PRISM-III score | 1.05 (95% CI: 1.01, 1.09) | 0.003 |
| PERSEVERENCE MODS prob. | 1.20 (95% CI: 1.07, 1.35) | 0.002 |

28-day mortality
Univariate model

| | | |
|---|---|---|
| PERSEVERENCE MODS prob. | 42.6 (7.0, 258.) | <0.001 |

Multivariate model

| | | |
|---|---|---|
| Age | 0.92 (95% CI: 0.84, 1.01) | 0.085 |
| PRISM-III score | 1.09 (95% CI: 1.04, 1.15) | <0.001 |
| PERSEVERENCE MODS prob. | 17.3 (95% CI: 2.4, 125.7) | 0.005 |

### Example 5

### Prognostic Enrichment of Organ Dysfunctions Using PERSEVEREnce Risk Models was Demonstrated

It was then tested whether simplified TreeNet® PERSEVEREnce models could result in meaningful enrichment of patients with death or persistent individual organ dysfunctions on day 7 of septic shock within the cohort. On reanalysis of the dataset, the PERSEVEREnce model correctly predicted those without death or day 7 MODS in 264 patients (true negatives) and incorrectly predicted the outcome of interest in 28 patients (false negatives). These true and false negative patients would be excluded from trials using PERSEVEREnce based classification. In an enriched cohort, 69.4% of subjects would be expected to have death or day 7 MODS relative to 34.5% without enrichment.

Using the PERSEVEREnce MODS model, the respective rates of cardiovascular, respiratory, renal, hepatic, hematologic, and neurologic dysfunctions in the enriched cohort, relative to one without enrichment would be 47.6 vs 24.3%, 62.8 vs 38.4%, 53.8 vs 28.4%, 37.2 vs 16.5%, 43.8 vs 21.3%, 17.6 vs 8.2%. The percent of patients requiring vasoactive support, mechanical ventilation, and renal replacement therapy in enriched vs. unenriched cohorts would be 44.3 vs 22.7%, 65.7 vs 40.7%, and 29.5 vs 14.2% respectively. Organ-specific PERSEVEREnce models had comparable rates of enrichment for cardiovascular, respiratory, and renal dysfunction. However, enrichment to 59.2%, 59.1%, and 29.7% can be achieved for hepatic, hematologic, and neurologic dysfunctions respectively.

### Example 6

### PERSEVEREnce was Used to Estimate Risk of Death or Persistent Organ Dysfunction on Day 7 of Septic Shock

The newly derived PERSEVEREnce biomarker risk models described in the preceding examples can be used to reliably estimate the risk of death or persistent organ dysfunctions in a cohort (e.g., large derivation cohorts, etc.) of pediatric septic shock patients. This is the first study to integrate whole blood/leukocyte and endothelial derived biomarkers to predict sepsis associated organ dysfunctions among children. Although 22 clinical and biological variables were considered during model development, 6 variables, based on 7 serum biomarkers (IL-8, HSP70, ICAM-1, Thrombomodulin, Angpt-1, Angpt-2, and Tie-2), were identified which contributed significantly to the predictive capabilities of the risk prediction models developed.

Interaction between IL-8 with ICAM-1 and Thrombomodulin with Angpt-2/Angpt-1 contributed to the predictive capabilities of the PERSEVEREnce model to estimate risk of death or day 7 MODS. IL-8 and ICAM-1 are secreted by leukocytes and endothelial cells. They are involved in neutrophil adhesion, extravasation, degranulation and contribute to endothelial dysfunction in sepsis [10,24]. Thrombomodulin is expressed by endothelial cells and inhibits coagulation under normal circumstances [25]. An increase in Angiopoietin-2 relative to Angiopoietin-1, is thought to result in inhibition of Thrombomodulin, and has been demonstrated to drive a procoagulant phenotype of endothelial cells [26]. Such recapitulation of key processes central to the pathophysiology of organ dysfunctions through agnostic machine learning algorithms lends biological plausibility to our risk prediction models. The relative importance of variables and their interactions varied in the organ specific PERSEVEREnce models, which reflects the unique interaction of activated leukocytes with the organotypic endothelium and highlights the complex biology of organ dysfunctions in sepsis.

PERSEVERE biomarkers can be used to retrospectively risk stratify patients and conduct secondary analyses of the randomized interventional trial of stress hydrocortisone in pediatric septic shock (SHIPPS, NCT03401398). Small scale studies have demonstrated correlation between and feasibility of measuring PERSEVERE and endothelial markers among pediatric septic shock patients using MicroKine assays within 20 minutes [20]. The PERSEVEREnce risk prediction models developed herein can therefore be translated to enrich patients for adaptive trials of repurposed or novel sepsis therapeutics.

Based on the data described herein, a 2-fold enrichment in death or day 7 MODS, cardiovascular, respiratory, and renal dysfunctions can be achieved with the PERSEVEREnce biomarker model. The organ-specific PERSEVEREnce models can be expected to yield an over 3-fold enrichment for hepatic, hematologic, and neurological dysfunctions. Because of the high rate of cardiovascular, respiratory, renal dysfunction and interventions used to support these organ systems within the cohort, the respective organ-specific models did not yield further enrichment beyond that offered by the model used to estimate risk of death or day 7 MODS.

The evolution of organ dysfunctions is dynamic [27]. PERSEVEREnce models were developed based on day 1 biomarkers to predict death or persistent organ dysfunction on day 7 with a high negative predictive value (NPV). It is plausible that biomarkers measured later in the sepsis course could result in a temporal reclassification of risk. Artificial intelligence models based on clinical and laboratory data have recently shown promise in identifying patients at risk of MODS in critically ill children with a high positive predictive value [28]. As such these efforts may be viewed as complementary and, if prospectively validated, can be deployed either concurrently or sequentially to recalibrate risk of organ dysfunctions over time.

The various methods and techniques described above provide a number of ways to carry out the disclosure. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the disclosure extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the disclosure. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Weiss S L, Fitzgerald J C, Pappachan J et al. Global Epidemiology of Pediatric Severe Sepsis: The Sepsis Prevalence, Outcomes, and Therapies Study. Am J Respir Crit Care Med 2015; 191:1147-57.
2. Zimmerman J J, Banks R, Berg R A et al. Critical Illness Factors Associated With Long-Term Mortality and Health Related Quality of Life Morbidity Following Community-Acquired Pediatric Septic Shock. Crit Care Med 2020; 48:319-28.
3. Carlton E F, Donnelly J P, Hensley M K et al. New Medical Device Acquisition During Pediatric Severe Sepsis Hospitalizations. Crit Care Med 2020; 48:725-31.
4. Iwashyna T J, Ely E W, Smith D M et al. Long-term cognitive impairment and functional disability among survivors of severe sepsis. JAMA 2010; 304:1787-94.
5. Weiss S L, Peters M J, Alhazzani W et al. Surviving Sepsis Campaign International Guidelines for the Management of Septic Shock and Sepsis-Associated Organ Dysfunction in Children. Pediatric Critical Care Medicine 2020; 21:e52.
6. Marshall J C. Why have clinical trials in sepsis failed?Trends Mol Med 2014; 20:195-203.
7. Atreya M R, Wong H R. Precision medicine in pediatric sepsis. Curr Opin Pediatr 2019; 31:322-7.
8. Stanski N L, Wong H R. Prognostic and predictive enrichment in sepsis. Nat Rev Nephrol 2020; 16:20-31.
9. Lelubre C, Vincent J-L. Mechanisms and treatment of organ failure in sepsis. Nat Rev Nephrol 2018; 14:417-27.
10. Joffre J, Hellman J, Ince C et al. Endothelial Responses in Sepsis. Am J Respir Crit Care Med 2020; 202:361-70.
11. Wong H R, Cvijanovich N Z, Allen G L et al. Validation of a gene expression-based subclassification strategy for pediatric septic shock. Crit Care Med 2011; 39:2511-7.
12. Wong H R, Cvijanovich N Z, Anas N et al. Developing a clinically feasible personalized medicine approach to pediatric septic shock. Am J Respir Crit Care Med 2015; 191:309-15.
13. Wong H R, Salisbury S, Xiao Q et al. The pediatric sepsis biomarker risk model. Crit Care 2012; 16:R174.
14. Wong H R, Cvijanovich N Z, Anas N et al. PERSEVERE-II: Redefining the pediatric sepsis biomarker risk model with septic shock phenotype. Crit Care Med 2016; 44:2010-7.
15. Wong H R, Caldwell J T, Cvijanovich N Z et al. Prospective clinical testing and experimental validation of the Pediatric Sepsis Biomarker Risk Model. Sci Transl Med 2019; 11, DOI: 10.1126/scitranslmed.aax9000.
16. Stanski N L, Stenson E K, Cvijanovich N Z et al. PERSEVERE Biomarkers Predict Severe Acute Kidney Injury and Renal Recovery in Pediatric Septic Shock. Am J Respir Crit Care Med 2020; 201:848-55.
17. Lautz A J, Wong H R, Ryan T D et al. Pediatric Sepsis Biomarker Risk Model Biomarkers and Estimation of Myocardial Dysfunction in Pediatric Septic Shock. Pediatr Crit Care Med 2021, DOI: 10.1097/PCC.0000000000002830.
18. Yehya N, Wong H R. Adaptation of a Biomarker-based Sepsis Mortality Risk Stratification Tool for Pediatric Acute Respiratory Distress Syndrome. Crit Care Med 2018; 46:e9-16.
19. Xing K, Murthy S, Liles W C et al. Clinical utility of biomarkers of endothelial activation in sepsis—a systematic review. Crit Care 2012; 16:R7.
20. Carlton E F, McHugh W M, McDonough K et al. Markers of Endothelial Dysfunction and Cytokines in High-Risk Pediatric Patients with Severe Sepsis. Am J Respir Crit Care Med 2020; 201:380-4.
21. Goldstein B, Giroir B, Randolph A et al. International pediatric sepsis consensus conference: definitions for sepsis and organ dysfunction in pediatrics. Pediatr Crit Care Med 2005; 6:2-8.
22. Pollack M M, Patel K M, Ruttimann U E. The Pediatric Risk of Mortality III—Acute Physiology Score (PRISM III-APS): a method of assessing physiologic instability for pediatric intensive care unit patients. J Pediatr 1997; 131:575-81.
23. McIntosh A M, Tong S, Deakyne S J et al. Validation of the Vasoactive-Inotropic Score in Pediatric Sepsis*. Pediatr Crit Care Med 2017; 18:750-7.
24. Le K T T, Chu X, Jaeger M et al. Leukocyte-Released Mediators in Response to Both Bacterial and Fungal Infections Trigger IFN Pathways, Independent of IL-1 and TNF-α, in Endothelial Cells. Frontiers in Immunology 2019; 10.
25. Giri H, Panicker S R, Cai X et al. Thrombomodulin is essential for maintaining quiescence in vascular endothelial cells. Proceedings of the National Academy of Sciences 2021; 118:e2022248118.
26. Daly C, Qian X, Castanaro C et al. Angiopoietins bind thrombomodulin and inhibit its function as a thrombin cofactor. Sci Rep 2018; 8:505.
27. Marshall J C, Deutschman C S. The Multiple Organ Dysfunction Syndrome: Syndrome, Metaphor, and Unsolved Clinical Challenge. Crit Care Med 2021; 49:1402-13.
28. Bose S N, Greenstein J L, Fackler J C et al. Early Prediction of Multiple Organ Dysfunction in the Pediatric Intensive Care Unit. Front Pediatr 2021; 9:711104.
29. Carcillo J A, Podd B, Aneja R et al. Pathophysiology of Pediatric Multiple Organ Dysfunction Syndrome. Pediatr Crit Care Med 2017; 18:532-45.
30. Kellum J A, Lameire N, Aspelin P et al. Kidney disease: Improving global outcomes (KDIGO) acute kidney injury work group. KDIGO clinical practice guideline for acute kidney injury. Kidney International Supplements 2012; 2:1-138.
31. Schlapbach L J, Weiss S L, Bembea M M et al. Scoring Systems for Organ Dysfunction and Multiple Organ Dysfunction: The PODIUM Consensus Conference. Pediatrics 2022; 149:S23-31.

What is claimed is:

1. A computer-implemented method of classifying a patient with septic shock as high risk of multiple organ dysfunction syndrome (MODS) and/or mortality or other than high risk of MODS and/or mortality, the method comprising:
receiving a sample from a pediatric patient with septic shock at a first time point;
analyzing the sample to determine expression levels of two or more biomarkers selected from the group consisting of: IL-8, HSP70, ICAM-1, Thrombomodulin, Angpt-2/Angpt-1, and Angpt-2/Tie-2;
determining whether the expression levels of each of the at least two biomarkers are greater than a respective cut-off expression level; and
classifying the patient as high risk of multiple organ dysfunction syndrome (MODS) and/or mortality, or other than high risk of MODS and/or mortality, based on the determination of whether the expression levels of each of the at least two biomarkers are greater than the respective cut-off expression level.

2. The method of claim 1, wherein a classification of high risk of MODS and/or mortality comprises:
a) a non-elevated level of ICAM-1, and an elevated level of IL-8;
b) an elevated level of ICAM-1, a non-elevated level of Angpt-2/Tie-2, and an elevated level of Thrombomodulin; or
c) an elevated level of ICAM-1, and an elevated level of Angpt-2/Tie-2;
and wherein a classification of other than high risk of MODS and/or mortality comprises:
d) a non-elevated level of ICAM-1, a non-elevated level of IL-8, a non-elevated level of Angpt-2/Angpt-1, and a non-elevated level of HSP70;
e) a non-elevated level of ICAM-1, a non-elevated level of IL-8, a non-elevated level of Angpt-2/Angpt-1, and an elevated level of HSP70;
f) a non-elevated level of ICAM-1, a non-elevated level of IL-8, and an elevated level of Angpt-2/Angpt-1; or
g) an elevated level of ICAM-1, a non-elevated level of Angpt-2/Tie-2, and a non-elevated level of Thrombomodulin.

3. The method of any preceding claim, wherein biomarker expression levels are determined by quantification of serum protein biomarker concentrations.

4. The method of any preceding claim, wherein biomarker expression levels are determined by concentrations and/or by cycle threshold (CT) values.

5. The method of any preceding claim, wherein the determined biomarker expression levels comprise expression levels of one or more pairs of biomarkers selected from the group consisting of: ICAM-1 and IL-8; ICAM-1 and Angpt-2/Tie-2; Angpt-2/Tie-2 and Thrombomodulin; IL-8 and Angpt-2/Angpt-1; and Angpt-2/Angpt-1 and HSP70.

6. The method of any preceding claim, wherein the determined biomarker expression levels comprise expression levels of three or more selected from the group consisting of: IL-8, HSP70, ICAM-1, Thrombomodulin, Angpt-2/Angpt-1, and/or Angpt-2/Tie-2.

7. The method of any preceding claim, wherein the determined biomarker expression levels comprise expression levels of a trio of biomarkers selected from the group consisting of: ICAM-1, IL-8, and Angpt-2/Angpt-1; IL-8, Angpt-2/Angpt-1, and HSP70; and ICAM-1, Angpt-2/Tie-2, and Thrombomodulin.

8. The method of any preceding claim, wherein the determined biomarker expression levels comprise expression levels of IL-8, HSP70, ICAM-1, Thrombomodulin, Angpt-2/Angpt-1, and Angpt-2/Tie-2.

9. The method of any preceding claim, wherein biomarker levels are determined by serum protein biomarker concentration, and wherein:
a) an elevated level of IL-8 corresponds to a serum IL-8 concentration greater than 3.66 log 10 fold change;
b) an elevated level of HSP70 corresponds to a serum HSP70 concentration greater than 6.32 log 10 fold change;
c) an elevated level of ICAM-1 corresponds to a serum ICAM-1 concentration greater than 5.89 log 10 fold change;
d) an elevated level of Thrombomodulin corresponds to a serum Thrombomodulin concentration greater than 3.94 log 10 fold change;
e) an elevated level of Angpt-2/Angpt-1 ratio corresponds to a serum Angpt-2/Angpt-1 ratio greater than 0.45; and
f) an elevated level of Angpt-2/Tie-2 corresponds to a serum Angpt-2/Tie-2 ratio greater than 1.06.

10. The method of any preceding claim, wherein the determination of whether the levels of the at least two biomarkers are non-elevated above a cut-off level comprises applying the biomarker expression level data to a decision tree comprising the two or more biomarkers.

11. The method of claim 10, comprising application of the decision tree of FIG. 9.

12. The method of any preceding claim, wherein a classification other than high risk comprises a classification of low risk or intermediate risk.

13. The method of any preceding claim, wherein MODS comprises cardiovascular, respiratory, renal, hepatic, hematologic, and/or neurologic dysfunction.

14. The method of claim 13, wherein MODS comprises cardiovascular dysfunction.

15. The method of any preceding claim, wherein MODS comprises dysfunction in one or more organs selected from heart, lungs, kidneys, liver, blood, and brain.

16. The method of any preceding claim, wherein high risk of MODS and/or mortality by day 7 of septic shock or other than high risk of MODS and/or mortality by day 7 of septic shock is determined.

17. The method of any preceding claim, wherein the classification is combined with one or more patient demographic data and/or clinical characteristics and/or results from other tests or indicia of septic shock and/or one or more additional biomarkers.

18. The method of claim 17, wherein the one or more additional biomarkers is selected from the group consisting of: heat shock protein 70 kDa 1B (HSPA1B), C-C Chemokine ligand 3 (CCL3), C-C Chemokine ligand 4 (CCL4), Granzyme B (GZMB), Interleukin-1 α (IL-1a), Matrix metallopeptidase 8 (MMP8), Angiopoietin-1 (Angpt-1), Angiopoietin-2 (Angpt-2), Tyrosine kinase with immunoglobulin-like loops and epidermal growth factor homology domains-2 (Tie-2), Vascular cell adhesion molecule-1 (VCAM-1), P-selectin, E-selectin, and Platelet and endothelial cell adhesion molecule-1 (PECAM-1).

19. The method of claim 17, wherein the patient demographic data and/or clinical characteristics and/or results from other tests or indicia of septic shock comprise at least one selected from the group consisting of: the septic shock causative organism, the presence or absence or chronic disease, and/or the age, gender, race, and/or co-morbidities of the patient.

20. The method of any preceding claim, wherein the classification is combined with one or more additional population-based risk scores.

21. The method of claim 20, wherein the one or more population-based risk scores comprises at least one selected from the group consisting of: Pediatric Sepsis Biomarker Risk Model (PERSEVERE), Pediatric Sepsis Biomarker Risk Model II (PERSEVERE II), Pediatric Risk of Mortality (PRISM), PRISM III, Pediatric Index of Mortality (PIM), and Pediatric Logistic Organ Dysfunction (PELOD).

22. The method of any preceding claim, wherein the sample is obtained within the first hour of presentation with septic shock.

23. The method of any preceding claim, wherein the sample is obtained within the first 24 hours, 48 hours, or 72 hours of presentation with septic shock.

24. The method of any preceding claim, further comprising administering a treatment comprising one or more high risk therapy to a patient that is classified as high risk, or administering a treatment excluding a high risk therapy to a patient that is not high risk, or to provide a method of treating a pediatric patient with septic shock.

25. The method of claim 24, wherein the one or more high risk therapy comprises at least one selected from the group consisting of: biological and/or immune enhancing therapy, extracorporeal membrane oxygenation/life support, plasmapheresis, pulmonary artery catheterization, high volume continuous hemofiltration, adjuvant hemoperfusion, extracorporeal hemadsorption, and/or plasma filtration and/or adsorption therapies.

26. The method of claim 25, wherein the biological and/or immune enhancing therapy comprises administration of GM-CSF, Interleukin-1 receptor antagonist, Interleukin-6 antagonist, anti-PD-1, recombinant thrombomodulin, Angiopoietin-2 inhibitors, and/or Angiopoietin-1 or Tie-2 agonist, and/or anti-PD-1.

27. The method of any preceding claim, wherein the patient is enrolled in a clinical trial.

28. The method of claim 27, wherein the patient is classified as high risk.

29. The method of claim 28, wherein the method comprises prognostic enrichment through enrollment of the high risk patient in the clinical trial.

30. The method of claim 29, further comprising administering a treatment comprising one or more high risk therapy to the patient in the clinical trial.

31. The method of claim 24, comprising improving an outcome in a pediatric patient with septic shock.

32. The method of claim 24, further comprising:
receiving a second sample from the treated patient at a second time point;
analyzing the second sample to determine the expression levels of two or more biomarkers comprising IL-8, HSP70, ICAM-1, Thrombomodulin, Angpt-2/Angpt-1, and/or Angpt-2/Tie-2;
determining whether the biomarker expression levels of each of the biomarkers are greater than a respective cut-off biomarker expression level;
classifying the patient as high risk of multiple organ dysfunction syndrome (MODS) and/or mortality, or other than high risk of MODS and/or mortality, based on the determination of whether the expression levels of each of the biomarkers are greater than the respective cut-off expression level;
maintaining the treatment being administered if the patient's high risk classification has not changed, or changing the treatment being administered if the patient's high risk classification has changed.

33. The method of claim 32, wherein the second time point is at least 18 hours after the first time point.

34. The method of claim 33, wherein the second time point is in the range of 24 to 96 hours, or longer, after the first time point.

35. The method of claim 33, wherein the second time point is about 1 day, 2 days, 3 days, or longer, after the first time point.

36. The method of claim 35, wherein the second time point is about 2 days after the first time point.

37. The method of claim 33, wherein the first time point is at day 1, wherein day 1 is within 24 hours of a septic shock diagnosis, and the second time point is at day 3.

38. The method of claim 33, wherein the first time point is within 24, 48, or 72 hours of a septic shock diagnosis, and the second time point is 1, 2, or 3 days after the first time point.

39. The method of claim 32, wherein a patient classified as high risk after the second time point is administered one or more high risk therapy.

40. The method of claim 39, wherein the one or more high risk therapy comprises at least one selected from the group consisting of: biological and/or immune enhancing therapy, extracorporeal membrane oxygenation/life support, plasmapheresis, pulmonary artery catheterization, high volume continuous hemofiltration, adjuvant hemoperfusion, adjuvant hemoperfusion, extracorporeal hemadsorption, and/or plasma filtration and/or adsorption therapies.

41. The method of claim 40, wherein the one or more high risk therapy comprises a biological and/or immune enhancing therapy.

42. The method of claim 32, wherein a patient not classified as high risk after the second time point is administered a treatment excluding a high risk therapy.

43. The method of claim 32, wherein the patient classified as high risk and administered one or more high risk therapy after the first time point is not classified as high risk after the second time point.

44. The method of any preceding claim, as part of a companion diagnostic or a point of care device or kit.

45. A diagnostic kit, test, or array comprising a reporter hybridization probe, and a capture hybridization probe specific for each of two or more mRNA, DNA, or protein biomarkers selected from the group consisting of: IL-8, HSP70, ICAM-1, Thrombomodulin, Angpt-2/Angpt-1, and Angpt-2/Tie-2.

46. The diagnostic kit, test, or array of claim 45, wherein the biomarkers comprise three or more selected from the group consisting of: IL-8, HSP70, ICAM-1, Thrombomodulin, Angpt-2/Angpt-1, and Angpt-2/Tie-2.

47. The diagnostic kit, test, or array of claim 46, wherein the biomarkers comprise IL-8, HSP70, ICAM-1, Thrombomodulin, Angpt-2/Angpt-1, and Angpt-2/Tie-2.

48. The diagnostic kit, test, or array of claim 47, further comprising a collection cartridge for immobilization of the hybridization probes.

49. The diagnostic kit, test, or array of claim 45, wherein the reporter and the capture hybridization probes comprise signal and barcode elements, respectively.

50. An apparatus or processing device suitable for detecting two or more biomarkers selected from the group consisting of: IL-8, HSP70, ICAM-1, Thrombomodulin, Angpt-2/Angpt-1, and Angpt-2/Tie-2.

51. The apparatus or processing device of claim 50, wherein the biomarkers comprise three or more selected from the group consisting of: IL-8, HSP70, ICAM-1, Thrombomodulin, Angpt-2/Angpt-1, and Angpt-2/Tie-2.

52. The apparatus or processing device of claim 51, wherein the biomarkers comprise IL-8, HSP70, ICAM-1, Thrombomodulin, Angpt-2/Angpt-1, and Angpt-2/Tie-2.

53. A composition comprising a reporter hybridization probe, and a capture hybridization probe specific for each of two or more biomarkers selected from the group consisting of: IL-8, HSP70, ICAM-1, Thrombomodulin, Angpt-2/Angpt-1, and Angpt-2/Tie-2.

54. The composition of claim 53, wherein the biomarkers comprise three or more selected from the group consisting of: IL-8, HSP70, ICAM-1, Thrombomodulin, Angpt-2/Angpt-1, and Angpt-2/Tie-2.

55. The composition of claim 54, wherein the biomarkers comprise IL-8, HSP70, ICAM-1, Thrombomodulin, Angpt-2/Angpt-1, and Angpt-2/Tie-2.
